SEQUENCE LISTING (127 sequences)

SEQ ID NO: 1 - protein, 185 aa, Mus musculus
MRLLIFLGLLWSLVATLLGSKWPEPVFGRLVSPGFPEKYADHQDRSWTLTAPPGYRLRLYFTHFDLELSYRCEYDFVKLSSGTKVLATLCGQESTDTEQAPGNDTFYSLGPSLKVTFHSDYSNEKPFTGFEAFYAAEDVDECRVSLGDSVPCDHYCHNYLGGYYCSCRAGYVLHQNKHTCSEQSL

SEQ ID NO: 2 - protein, 244 aa, Mus musculus
MSIFTSFLLLCVVTVVYAETLTEGVQNSCPVVTCSSPGLNGFPGKDGRDGAKGEKGEPGQGLRGLQGPPGKVGPTGPPGNPGLKGAVGPKGDRGDRAEFDTSEIDSEIAALRSELRALRNWVLFSLSEKVGKKYFVSSVKKMSLDRVKALCSEFQGSVATPRNAEENSAIQKVAKDIAYLGITDVRVEGSFEDLTGNRVRYTNWNDGEPNNTGDGEDCVVILGNGKWNDVPCSDSFLAICEFSD

SEQ ID NO: 3 - protein, 239 aa, Mus musculus
MLLLPLLPVLLCVVSVSSSGSQTCEDTLKTCSVIACGRDGRDGPKGEKGEPGQGLRGLQGPPGKLGPPGSVGSPGSPGPKGQKGDHGDNRAIEEKLANMEAEIRILKSKLQLTNKLHAFSMGKKSGKKLFVTNHEKMPFSKVKSLCTELQGTVAIPRNAEENKAIQEVATGIAFLGITDEATEGQFMYVTGGRLTYSNWKKDEPNNHGSGEDCVIILDNGLWNDISCQASFKAVCEFPA

SEQ ID NO: 4 - protein, 652 aa, Homo sapiens
MATSMGLLLLLLLLLTQPGAGTGADTEAVVCVGTACYTAHSGKLSAAEAQNHCNQNGGNLATVKSKEEAQHVQRVLAQLLRREAALTARMSKFWIGLQREKGKCLDPSLPLKGFSWVGGGEDTPYSNWHKELRNSCISKRCVSLLLDLSQPLLPSRLPKWSEGPCGSPGSPGSNIEGFVCKFSFKGMCRPLALGGPGQVTYTTPFQTTSSSLEAVPFASAANVACGEGDKDETQSHYFLCKEKAPDVFDWGSSGPLCVSPKYGCNFNNGGCHQDCFEGGDGSFLCGCRPGFRLLDDLVTCASRNPCSSSPCRGGATCVLGPHGKNYTCRCPQGYQLDSSQLDCVDVDECQDSPCAQECVNTPGGFRCECWVGYEPGGPGEGACQDVDECALGRSPCAQGCTNTDGSFHCSCEEGYVLAGEDGTQCQDVDECVGPGGPLCDSLCFNTQGSFHCGCLPGWVLAPNGVSCTMGPVSLGPPSGPPDEEDKGEKEGSTVPRAATASPTRGPEGTPKATPTTSRPSLSSDAPITSAPLKMLAPSGSPGVWREPSIHHATAASGPQEPAGGDSSVATQNNDGTDGQKLLLFYILGTVVAILLLLALALGLLVYRKRRAKREEKKEKKPQNAADSYSWVPERAESRAMENQYSPTPGTDC

SEQ ID NO: 5 - protein, 644 aa, Mus musculus
MAISTGLFLLLGLLGQPWAGAAADSQAVVCEGTACYTAHWGKLSAAEAQHRCNENGGNLATVKSEEEARHVQQALTQLLKTKAPLEAKMGKFWIGLQREKGNCTYHDLPMRGFSWVGGGEDTAYSNWYKASKSSCIFKRCVSLILDLSLTPHPSHLPKWHESPCGTPEAPGNSIEGFLCKFNFKGMCRPLALGGPGRVTYTTPFQATTSSLEAVPFASVANVACGDEAKSETHYFLCNEKTPGIFHWGSSGPLCVSPKFGCSFNNGGCQQDCFEGGDGSFRCGCRPGFRLLDDLVTCASRNPCSSNPCTGGGMCHSVPLSENYTCRCPSGYQLDSSQVHCVDIDECQDSPCAQDCVNTLGSFHCECWVGYQPSGPKEEACEDVDECAAANSPCAQGCINTDGSFYCSCKEGYIVSGEDSTQCEDIDECSDARGNPCDSLCFNTDGSFRCGCPPGWELAPNGVFCSRGTVFSELPARPPQKEDNDDRKESTMPPTEMPSSPSGSKDVSNRAQTTGLFVQSDIPTASVPLEIEIPSEVSDVWFELGTYLPTTSGHSKPTHEDSVSAHSDTDGQNLLLFYILGTVVAISLLLVLALGILIYHKRRAKKEEIKEKKPQNAADSYSWVPERAESQAPENQYSPTPGTDC

SEQ ID NO: 6 - protein, 688 aa, Homo sapiens
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 7 - protein, 652 aa, Homo sapiens
MATSMGLLLLLLLLLTQPGAGTGADTEAVVCVGTACYTAHSGKLSAAEAQNHCNQNGGNLATVKSKEEAQHVQRVLAQLLRREAALTARMSKFWIGLQREKGKCLDPSLPLKGFSWVGGGEDTPYSNWHKELRNSCISKRCVSLLLDLSQPLLPNRLPKWSEGPCGSPGSPGSNIEGFVCKFSFKGMCRPLALGGPGQVTYTTPFQTTSSSLEAVPFASAANVACGEGDKDETQSHYFLCKEKAPDVFDWGSSGPLCVSPKYGCNFNNGGCHQDCFEGGDGSFLCGCRPGFRLLDDLVTCASRNPCSSSPCRGGATCVLGPHGKNYTCRCPQGYQLDSSQLDCVDVDECQDSPCAQECVNTPGGFRCECWVGYEPGGPGEGACQDVDECALGRSPCAQGCTNTDGSFHCSCEEGYVLAGEDGTQCQDVDECVGPGGPLCDSLCFNTQGSFHCGCLPGWVLAPNGVSCTMGPVSLGPPSGPPDEEDKGEKEGSTVPRAATASPTRGPEGTPKATPTTSRPSLSSDAPITSAPLKMLAPSGSSGVWREPSIHHATAASGPQEPAGGDSSVATQNNDGTDGQKLLLFYILGTVVAILLLLALALGLLVYRKRRAKREEKKEKKPQNAADSYSWVPERAESRAMENQYSPTPGTDC

SEQ ID NO: 8 - protein, 248 aa, Homo sapiens
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 9 - protein, 248 aa, Homo sapiens; identical to SEQ ID NO: 8

SEQ ID NO: 10 - protein, 643 aa, Rattus norvegicus
MVTSTGLLLLLGLLGQLWAGAAADSEAVVCEGTACYTAHWGKLSAAEAQHRCNENGGNLATVKSEEEARHVQEALAQLLKTKAPSETKIGKFWIGLQREKGKCTYHDLPMKGFSWVGGGEDTTYSNWYKASKSSCISKRCVSLILDLSLKPHPSHLPKWHESPCGTPDAPGNSIEGFLCKFNFKGMCSPLALGGPGQLTYTTPFQATTSSLKAVPFASVANVVCGDEAESKTNYYLCKETTAGVFHWGSSGPLCVSPKFGCSFNNGGCQQDCFEGGDGSFRCGCRPGFRLLDDLVTCASRNPCSSNPCTGGGMCHSVPLSENYTCHCPRGYQLDSSQVHCVDIDECEDSPCDQECINTPGGFHCECWVGYQSSGSKEEACEDVDECTAAYSPCAQGCTNTDGSFYCSCKEGYIMSGEDSTQCEDIDECLGNPCDTLCINTDGSFRCGCPAGFELAPNGVSCTRGSMFSELPARPPQKEDKGDGKESTVPLTEMPGSLNGSKDVSNRAQTTDLSIQSDSSTASVPLEIEVSSEASDVWLDLGTYLPTTSGHSQPTHEDSVPAHSDSDTDGQKLLLFYILGTVVAISLLLALALGLLIYLKRKAKKEEIKEKKAQNAADSYSWIPERAESRAPENQYSPTPGTDC

SEQ ID NO: 11 - protein, 686 aa, Homo sapiens
MRLLTLLGLLCGSVATPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSALCSGQVFTQRSGELSSPEYPRPYPKLSSCTYSISLEEGFSVILDFVESFDVETHPETLCPYDFLKIQTDREEHGPFCGKTLPHRIETKSNTVTITFVTDESGDHTGWKIHYTSTAQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 12 - protein, 185 aa, Homo sapiens
MRLLTLLGLLCGSVATPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSEQSL

SEQ ID NO: 13 - protein, 728 aa, Homo sapiens
MRWLLLYYALCFSLSKASAHTVELNNMFGQIQSPGYPDSYPSDSEVTWNITVPDGFRIKLYFMHFNLESSYLCEYDYVKVETEDQVLATFCGRETTDTEQTPGQEVVLSPGSFMSITFRSDFSNEERFTGFDAHYMAVDVDECKEREDEELSCDHYCHNYIGGYYCSCRFGYILHTDNRTCRVECSDNLFTQRTGVITSPDFPNPYPKSSECLYTIELEEGFMVNLQFEDIFDIEDHPEVPCPYDYIKIKVGPKVLGPFCGEKAPEPISTQSHSVLILFHSDNSGENRGWRLSYRAAGNECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPECGQPSRSLPSLVKRIIGGRNAEPGLFPWQALIVVEDTSRVPNDKWFGSGALLSASWILTAAHVLRSQRRDTTVIPVSKEHVTVYLGLHDVRDKSGAVNSSAARVVLHPDFNIQNYNHDIALVQLQEPVPLGPHVMPVCLPRLEPEGPAPHMLGLVAGWGISNPNVTVDEIISSGTRTLSDVLQYVKLPVVPHAECKTSYESRSGNYSVTENMFCAGYYEGGKDTCLGDSGGAFVIFDDLSQRWVVQGLVSWGGPEECGSKQVYGVYTKVSNYVDWVWEQMGLPQSVVEPQVER

SEQ ID NO: 14 - protein, 699 aa, Homo sapiens
MRWLLLYYALCFSLSKASAHTVELNNMFGQIQSPGYPDSYPSDSEVTWNITVPDGFRIKLYFMHFNLESSYLCEYDYVKVETEDQVLATFCGRETTDTEQTPGQEVVLSPGSFMSITFRSDFSNEERFTGFDAHYMAVDVDECKEREDEELSCDHYCHNYIGGYYCSCRFGYILHTDNRTCRVECSDNLFTQRTGVITSPDFPNPYPKSSECLYTIELEEGFMVNLQFEDIFDIEDHPEVPCPYDYIKIKVGPKVLGPFCGEKAPEPISTQSHSVLILFHSDNSGENRGWRLSYRAAGNECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPVCGLPKFSRKLMARIFNGRPAQKGTTPWIAMLSHLNGQPFCGGSLLGSSWIVTAAHCLHQSLDPEDPTLRDSDLLSPSDFKIILGKHWRLRSDENEQHLGVKHTTLHPQYDPNTFENDVALVELLESPVLNAFVMPICLPEGPQQEGAMVIVSGWGKQFLQRFPETLMEIEIPIVDHSTCQKAYAPLKKKVTRDMICAGEKEGGKDACAGDSGGPMVTLNRERGQWYLVGTVSWGDDCGKKDRYGVYSYIHHNKDWIQRVTGVRN

SEQ ID NO: 15 - protein, 239 aa, Mus musculus
  MISC_FEATURE (91)..(116): Unknown
MLLLPLLPVLLCVVSVSSSGSQTCEDTLKTCSVIACGRDGRDGPKGEKGEPGQGLRGLQGPPGKLGPPGSVGSPGSPGPKGQKGDHGDNRXXXXXXXXXXXXXXXXXXXXXXXXXXHAFSMGKKSGKKLFVTNHEKMPFSKVKSLCTELQGTVAIPRNAEENKAIQEVATGIAFLGITDEATEGQFMYVTGGRLTYSNWKKDEPNNHGSGEDCVIILDNGLWNDISCQASFKAVCEFPA

SEQ ID NO: 16 - protein, 185 aa, Oncorhynchus mykiss
MEKLAILLLLSASIALGDANLTQLLGLEPLLKTKVEQTTPEAQVEAVQEGIKEGSCPSDWYTYGSHCFKFVSIQQSFVDSEQNCLALGGNLASVHSLLEYQFMQALTKDANGHLHSTWLGGFDAIKEGTWMWSDGSRFDYTNWDTDEPNNAGEGEDCLHMNAASAKLWFDVPCEWKFASLCSRRM

SEQ ID NO: 17 - protein, 240 aa, Sus scrofa
MSLFPSLHLLLLIVMTASHTETENCEDIQNTCLVISCDSPGINGLPGKDGLDGAKGEKGEPGQGLIGLQGLPGMVGPQGSPGIPGLPGLKGQKGDSGIDPGNSLANLRSELDNIKKWLIFAQGKQVGKKLYLTNGKKMSFNGVKALCAQFQASVATPTNSRENQAIQELAGTEAFLGITDEYTEGQFVDLTGKRVRYQNWNDGEPNNADSAEHCVEILKDGKWNDIFCSSQLSAVCEFPA

SEQ ID NO: 18 - protein, 239 aa, Mus musculus; identical to SEQ ID NO: 3

SEQ ID NO: 19 - protein, 244 aa, Mus musculus; identical to SEQ ID NO: 2

SEQ ID NO: 20 - protein, 381 aa, Macaca nemestrina
MSDSKEPRLQQLDLLEEEQLGGVGFRQTRGYKSLAGCLGHGPLVLQLLSFTLLAGLLVQVSKVPSSLSQGQSKQDAIYQNLTQLKVAVSELSEKSKQQEIYQELTRLKAAVGELPEKSKQQEIYEELTRLRAAVGELPEKSKLQEIYQELTRLKAAVGELPEKSKQQEIYQELSRLKAAVGDLPEKSKQQEIYQKLTQLKAAVDGLPDRSKQQEIYQELIQLKAAVERLCHPCPWEWTFFQGNCYFMSNSQRNWHDSITACQEVGAQLVVIKSAEEQNFLQLQSSRSNRFTWMGLSDLNHEGTWQWVDGSPLLPSFKQYWNKGEPNNVGEEDCAEFSGNGWNDDKCNLAKFWICKKSAASCSGDEERLLSPAPTTPNPPPA

SEQ ID NO: 21 - protein, 246 aa, Carassius auratus
LLLLQFALQLLDGAEPQNLNCPAYGGVPGTPGHNGLPGRDGRDGKDGAIGPKGEKGESGVSVQGPPGKAGPPGTAGEKGERGPSGPQGSPGSESVLESLKSEIQQLKAKIATFEKVSSVCHFRKVGQKYYITDGVVGNFDQGLKSCMEFGGTMVSPRTSAENQALLKLVVSSGLGSKKPYIGVTDRKTEGQFVDTEGKQLTFTNWGPGQPDDYKGLQDCGVIEDTGLWDDGGCGDIRPIMCEIDIK

SEQ ID NO: 22 - protein, 251 aa, Danio rerio
MALLKLFLGALLLLQLVLQLMAGAADPQSLNCPAYAGVPGTPGHNGLPGRDGRVGRDGANGPKGEKGEPGVNVQGPPGKAGPPGPAGAKGERGPSGLPGQDCMSDSLKSELQKLSDKIALIEKVVNFKTFKKVGQKYYVTDDVEETFDKGMQYCSSNGGALVLPRTLEENALLKVFVSSAFKRLFIRITDREKEGEFVDTDRKKLTFTNWGPNQPDNYKGAQDCGAIADSGLWDDVSCDSLYPIICEIEIK

SEQ ID NO: 23 - protein, 256 aa, Cyprinus carpio
MALFKLFLGTLLLLQFALQLLDGAEPQNLNCPAYGGVPGTPGHNGLPGRDGRDGKDGAIGPKGEKGESGVSVQGPPGKAGPPGPAGEKGERGPTGSQGSPGSESVLESLKSEIQQLKAKIATFEKVASVGHFRQVGQKYYITDGVVGTFDQGLKFCKDFGGTMVFPRTSAENQALLKLVVSSGLSSKKPYIGVTDRETEGRFVNTEGKQLTFTNWGPGQPDDYKGLQDCGVIEDSGLWDDGSCGDIRPIMCEIDNK

SEQ ID NO: 24 - protein, 31 aa, Sus scrofa
TKGEKGEPGPGFRGSQGPPGKMGPPGNIGET

SEQ ID NO: 25 - protein, 254 aa, Gallus gallus
MTLLQPFSALLLCLSLMMATSLLTTDKPEEKMYSCPIIQCSAPAVNGLPGRDGRDGPKGEKGDPGEGLRGLQGLPGKAGPQGLKGEVGPQGEKGQKGERGIVVTDDLHRQITDLEAKIRVLEDDLSRYKKALSLKDVVNVGKKMFVSTGKKYNFEKGKSLCAKAGSVLASPRNEAENTALKDLIDPSSQAYIGISDAQTEGRFMYLSGGPLTYSNWKPGEPNNHKNEDCAVIEDSGKWNDLDCSNSNIFIICEL

SEQ ID NO: 26 - protein, 244 aa, Mus musculus; identical to SEQ ID NO: 2

SEQ ID NO: 27 - protein, 239 aa, Mus musculus; identical to SEQ ID NO: 3

SEQ ID NO: 28 - protein, 238 aa, Rattus norvegicus
MLLLPLLVLLCVVSVSSSGSQTCEETLKTCSVIACGRDGRDGPKGEKGEPGQGLRGLQGPPGKLGPPGSVGAPGSQGPKGQKGDRGDSRAIEVKLANMEAEINTLKSKLELTNKLHAFSMGKKSGKKFFVTNHERMPFSKVKALCSELRGTVAIPRNAEENKAIQEVAKTSAFLGITDEVTEGQFMYVTGGRLTYSNWKKDEPNDHGSGEDCVTIVDNGLWNDISCQASHTAVCEFPA

SEQ ID NO: 29 - protein, 244 aa, Rattus norvegicus
MSLFTSFLLLCVLTAVYAETLTEGAQSSCPVIACSSPGLNGFPGKDGHDGAKGEKGEPGQGLRGLQGPPGKVGPAGPPGNPGSKGATGPKGDRGESVEFDTTNIDLEIAALRSELRAMRKWVLLSMSENVGKKYFMSSVRRMPLNRAKALCSELQGTVATPRNAEENRAIQNVAKDVAFLGITDQRTENVFEDLTGNRVRYTNWNEGEPNNVGSGENCVVLLTNGKWNDVPCSDSFLVVCEFSD

SEQ ID NO: 30 - protein, 248 aa, Homo sapiens; identical to SEQ ID NO: 8

SEQ ID NO: 31 - protein, 329 aa, Homo sapiens
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVE

SEQ ID NO: 32 - protein, 248 aa, Homo sapiens
MSLFPSLPLLLLSMVAASYSETVTCEDAQKTCPAVIACSSPGINGFPGKDGCDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 33 - protein, 248 aa, Homo sapiens; identical to SEQ ID NO: 8

SEQ ID NO: 34 - protein, 248 aa, Homo sapiens; identical to SEQ ID NO: 8

SEQ ID NO: 35 - protein, 248 aa, Homo sapiens [sequence omitted]

SEQ ID NO: 36 - protein, 248 aa, Homo sapiens
MSLFPSLPLLLLSMVAASYSETVTCEDAQKTCPAVIACSSPGINGFPGKDGRDGTKEEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 37 - protein, 248 aa, Homo sapiens; identical to SEQ ID NO: 8

SEQ ID NO: 38 - protein, 248 aa, Homo sapiens; identical to SEQ ID NO: 8

SEQ ID NO: 39 - protein, 652 aa, Homo sapiens; identical to SEQ ID NO: 7

SEQ ID NO: 40 - protein, 251 aa, Danio rerio; identical to SEQ ID NO: 22

SEQ ID NO: 41 - protein, 118 aa, Cyprinus carpio
KIQTGSNTVSILFHSDNSGDNLGWKLTYTSTGSECSPLAAPLNGHLEPLQSNYIFKDHIMLTCDPGYSLRQGDKEFEHYQIECQRDGKWSSDVPLCKKKESQRRHRSLPSILTNQILS

SEQ ID NO: 42 - protein, 652 aa, Homo sapiens; identical to SEQ ID NO: 4

SEQ ID NO: 43 - protein, 742 aa, Mus musculus
MKDDFAEEEEVQSFGYKRFGIQEGTQCTKCKNNWALKFSIVLLYILCALLTITVAILGYKVVEKMDNVTDGMETSHQTYDNKLTAVESDLKKLGDQAGKKALSTNSELSTFRSDILDLRQQLQEITEKTSKNKDTLEKLQANGDSLVDRQSQLKETLQNNSFLITTVNKTLQAYNGYVTNLQQDTSVLQGNLQSQMYSQSVVIMNLNNLNLTQVQQRNLISNLQQSVDDTSLAIQRIKNDFQNLQQVFLQAKKDTDWLKEKVQSLQTLAANNSALAKANNDTLEDMNSQLSSFTGQMDNITTISQANEQSLKDLQDLHKDTENRTAVKFSQLEERFQVFETDIVNIISNISYTAHHLRTLTSNLNDVRTTCTDTLTRHTDDLTSLNNTLVNIRLDSISLRMQQDMMRSKLDTEVANLSVVMEEMKLVDSKHGQLIKNFTILQGPPGPRGPKGDRGSQGPPGPTGNKGQKGEKGEPGPPGPAGERGTIGPVGPPGERGSKGSKGSQGPKGSRGSPGKPGPQGPSGDPGPPGPPGKDGLPGPQGPPGFQGLQGTVGEPGVPGPRGLPGLPGVPGMPGPKGPPGPPGPSGAMEPLALQNEPTPASEVNGCPPHWKNFTDKCYYFSLEKEIFEDAKLFCEDKSSHLVFINSREEQQWIKKHTVGRESHWIGLTDSEQESEWKWLDGSPVDYKNWKAGQPDNWGSGHGPGEDCAGLIYAGQWNDFQCDEINNFICEKEREAVPSSIL

SEQ ID NO: 44 - protein, 321 aa, Bos taurus
MLPLPLSILLLLTQSQSFLGEEMDVYSEKTLTDPCTLVVCAPPADSLRGHDGRDGKEGPQGEKGDPGPPGMPGPAGREGPSGRQGSMGPPGTPGPKGEPGPEGGVGAPGMPGSPGPAGLKGERGTPGPGGAIGPQGPSGAMGPPGLKGDRGDPGEKGARGETSVLEVDTLRQRMRNLEGEVQRLQNIVTQYRKAVLFPDGQAVGEKIFKTAGAVKSYSDAEQLCREAKGQLASPRSSAENEAVTQLVRAKNKHAYLSMNDISKEGKFTYPTGGSLDYSNWAPGEPNNRAKDEGPENCLEIYSDGNWNDIECREERLVICEF

SEQ ID NO: 45 - protein, 371 aa, Bos taurus
MLLLPLSVLLLLTQPWRSLGAEMKIYSQKTLANGCTLVVCRPPEGGLPGRDGQDGREGPQGEKGDPGSPGPAGRAGRPGPAGPIGPKGDNGSAGEPGPKGDTGPPGPPGMPGPAGREGPSGKQGSMGPPGTPGPKGDTGPKGGMGAPGMQGSPGPAGLKGERGAPGELGAPGSAGVAGPAGAIGPQGPSGARGPPGLKGDRGDPGERGAKGESGLADVNALKQRVTILEGQLQRLQNAFSRYKKAVLFPDGQAVGKKIFKTAGAVKSYSDAQQLCREAKGQLASPRSAAENEAVAQLVRAKNNDAFLSMNDISTEGKFTYPTGESLVYSNWASGEPNNNNAGQPENCVQIYREGKWNDVPCSEPLLVICEF

SEQ ID NO: 46 - protein, 272 aa, Mus musculus
MMMRDLALAGMLISLAFLSLLPSGCPQQTTEDACSVQILVPGLKGDAGEKGDKGAPGRPGRVGPTGEKGDMGDKGQKGTVGRHGKIGPIGAKGEKGDSGDIGPPGPSGEPGIPCECSQLRKAIGEMDNQVTQLTTELKFIKNAVAGVRETESKIYLLVKEEKRYADAQLSCQARGGTLSMPKDEAANGLMASYLAQAGLARVFIGINDLEKEGAFVYSDRSPMQTFNKWRSGEPNNAYDEEDCVEMVASGGWNDVACHITMYFMCEFDKENL

SEQ ID NO: 47 - protein, 420 aa, Mus musculus
MNGFRVLLRSNLSMLLLLALLHFQSLGLDVDSRSAAEVCATHTISPGPKGDDGERGDTGEEGKDGKVGRQGPKGVKGELGDMGAQGNIGKSGPIGKKGDKGEKGLLGIPGEKGKAGTICDCGRYRKVVGQLDISVARLKTSMKFIKNVIAGIRETEEKFYYIVQEEKNYRESLTHCRIRGGMLAMPKDEVVNTLIADYVAKSGFFRVFIGVNDLEREGQYVFTDNTPLQNYSNWKEEEPSDPSGHEDCVEMLSSGRWNDTECHLTMYFVSSLQEDLIEDCLREQGLLVQVTPANQELLFGIDTFLGPMSCVYQRTGTKQKLYSQCRLWDGLAKKQTNETANIATFCKGAEPNRGSRPCGQKQEMMTLMMSGNKGITTFPESDNLFKWVGTMLGAAGTIDEDLKYKLSLNSPVVTLIIHPQ

SEQ ID NO: 48 - protein, 742 aa, Mus musculus; identical to SEQ ID NO: 43

SEQ ID NO: 49 - protein, 251 aa, Danio rerio; identical to SEQ ID NO: 22

SEQ ID NO: 50 - protein, 742 aa, Homo sapiens
MKDDFAEEEEVQSFGYKRFGIQEGTQCTKCKNNWALKFSIILLYILCALLTITVAILGYKVVEKMDNVTGGMETSRQTYDDKLTAVESDLKKLGDQTGKKAISTNSELSTFRSDILDLRQQLREITEKTSKNKDTLEKLQASGDALVDRQSQLKETLENNSFLITTVNKTLQAYNGYVTNLQQDTSVLQGNLQNQMYSHNVVIMNLNNLNLTQVQQRNLITNLQRSVDDTSQAIQRIKNDFQNLQQVFLQAKKDTDWLKEKVQSLQTLAANNSALAKANNDTLEDMNSQLNSFTGQMENITTISQANEQNLKDLQDLHKDAENRTAIKFNQLEERFQLFETDIVNIISNISYTAHHLRTLTSNLNEVRTTCTDTLTKHTDDLTSLNNTLANIRLDSVSLRMQQDLMRSRLDTEVANLSVIMEEMKLVDSKHGQLIKNFTILQGPPGPRGPRGDRGSQGPPGPTGNKGQKGEKGEPGPPGPAGERGPIGPAGPPGERGGKGSKGSQGPKGSRGSPGKPGPQGPSGDPGPPGPPGKEGLPGPQGPPGFQGLQGTVGEPGVPGPRGLPGLPGVPGMPGPKGPPGPPGPSGAVVPLALQNEPTPAPEDNGCPPHWKNFTDKCYYFSVEKEIFEDAKLFCEDKSSHLVFINTREEQQWIKKQMVGRESHWIGLTDSERENEWKWLDGTSPDYKNWKAGQPDNWGHGHGPGEDCAGLIYAGQWNDFQCEDVNNFICEKDRETVLSSAL

SEQ ID NO: 51 - protein, 622 aa, Homo sapiens
MKDDFAEEEEVQSFGYKRFGIQEGTQCTKCKNNWALKFSIILLYILCALLTITVAILGYKVVEKMDNVTGGMETSRQTYDDKLTAVESDLKKLGDQTGKKAISTNSELSTFRSDILDLRQQLREITEKTSKNKDTLEKLQASGDALVDRQSQLKETLENNSFLITTVNKTLQAYNGYVTNLQQDTSVLQGNLQNQMYSHNVVIMNLNNLNLTQVQQRNLITNLQRSVDDTSQAIQRIKNDFQNLQQVFLQAKKDTDWLKEKVQSLQTLAANNSALAKANNDTLEDMNSQLNSFTGQMENITTISQANEQNLKDLQDLHKDAENRTAIKFNQLEERFQLFETDIVNIISNISYTAHHLRTLTSNLNEVRTTCTDTLTKHTDDLTSLNNTLANIRLDSVSLRMQQDLMRSRLDTEVANLSVIMEEMKLVDSKHGQLIKNFTILQGPPGPRGPRGDRGSQGPPGPTGNKGQKGEKGEPGPPGPAGERGPIGPAGPPGERGGKGSKGSQGPKGSRGSPGKPGPQGPSGDPGPPGPPGKEGLPGPQGPPGFQGLQGTVGEPGVPGPRGLPGLPGVPGMPGPKGPPGPPGPSGAVVPLALQNEPTPAPEDNSKSKPSLQPGGQGSACA

SEQ ID NO: 52 - protein, 742 aa, Mus musculus
MKDDFAEEEEVQSFGYKRFGIHEGTQCTKCINNWALKFSIVLLYILCALLTITVAILGYKVVEKMDNVSDGMETSHQTYDNKLTAVESDLKKLGDQAGKKALSTNSELSTFRSDILDLRQQLQEITEKTSKNKDTLEKLQANGDSLVDRQSQLKETLQNNSFLITTVNKTLQAYNGYVTNLQQDTNVLQGNLQSQMYSQSVVIMNLNNLNLTQVQQRNLISNLQQSVDDTSLAIQRIKNDFQNLQQVFLQAKKDTDWLKEKVQSLQTLAANNSALAKANNDTLEDMNSQLSSFTGQMDNITTISQANEQSLKDLQDLHKDTENRTAVKFSQLEERFQVFETDIVNIISNISYTAHHLRTLTSNLNDVWTTCTDTLTRHTDDLTSLNNTLVNIRLDSISLRMQQDMMRSKLDTEVANLSVVMEEMKLVDSKHGQLIKNFTILQGPPGPRGPKGDRGSQGPPGPTGNKGQKGEKGEPGPPGPAGERGTIGPVGPPGERGSKGSKGSQGPKGSRGSPGKPGPQGPSGDPGPPGPPGKDGLPGPQGPPGFQGLQGTVGEPGVPGPRGLPGLPGVPGMPGPKGPPGPPGPSGAMEPLALQNEPTPASEVNGCPPHWKNFTDKCYYFSLEKEILEDAKLFCEDKSSHLVFINSREEQQWIKKHTVGRESHWIGLTDSEQESEWKWLDGSPVDYKNWKAGQPDNWGSGHGPGEDCAGLIYAGQWNDFQCDEINNFICEKEREAVPSSIL

SEQ ID NO: 53 - protein, 321 aa, Bos taurus; identical to SEQ ID NO: 44

SEQ ID NO: 54 - protein, 239 aa, Mus musculus; identical to SEQ ID NO: 3

SEQ ID NO: 55 - protein, 244 aa, Mus musculus; identical to SEQ ID NO: 2

SEQ ID NO: 56 - protein, 277 aa, Homo sapiens
MNGFASLLRRNQFILLVLFLLQIQSLGLDIDSRPTAEVCATHTISPGPKGDDGEKGDPGEEGKHGKVGRMGPKGIKGELGDMGDRGNIGKTGPIGKKGDKGEKGLLGIPGEKGKAGTVCDCGRYRKFVGQLDISIARLKTSMKFVKNVIAGIRETEEKFYYIVQEEKNYRESLTHCRIRGGMLAMPKDEAANTLIADYVAKSGFFRVFIGVNDLEREGQYMFTDNTPLQNYSNWNEGEPSDPYGHEDCVEMLSSGRWNDTECHLTMYFVCEFIKKKK

SEQ ID NO: 57 - protein, 742 aa, Homo sapiens; identical to SEQ ID NO: 50

SEQ ID NO: 58 - protein, 246 aa, Carassius auratus; identical to SEQ ID NO: 21

SEQ ID NO: 59 - protein, 251 aa, Danio rerio; identical to SEQ ID NO: 22

SEQ ID NO: 60 - protein, 256 aa, Cyprinus carpio; identical to SEQ ID NO: 23

SEQ ID NO: 61 - protein, 222 aa, Gallus gallus
MLSYSFCMIAAAVALLTPCHAQNCAGAPELPSIPGVSGLLGLGALKRYFGSLLWPYGEEKLPECQWLQRQQDLSTSSDDELGNVLLNLRQRILQLEGVLALDGKITKVGEKIFASNGKEVNFSSALESCEETGGTLATPMNEEENKAIMGIVKQYNRYAYLGIKESDTAGQFKYVNNQPLNYTSWQQYEPNGKGTEKCVEMYTDGNWKDRKCNLYRLTVCEY

SEQ ID NO: 62 - protein, 371 aa, Bos taurus
MLLLPLSVLLLLTQPWRSLGAEMTTFSQKILANACTLVMCSPLESGLPGHDGQDGRECPHGEKGDPGSPGPAGRAGRPGWVGPIGPKGDNGFVGEPGPKGDTGPRGPPGMPGPAGREGPSGKQGSMGPPGTPGPKGETGPKGGVGAPGIQGFPGPSGLKGEKGAPGETGAPGRAGVTGPSGAIGPQGPSGARGPPGLKGDRGDPGETGAKGESGLAEVNALKQRVTILDGHLRRFQNAFSQYKKAVLFPDGQAVGEKIFKTAGAVKSYSDAEQLCREAKGQLASPRSSAENEAVTQMVRAQEKNAYLSMNDISTEGRFTYPTGEILVYSNWADGEPNNSDEGQPENCVEIFPDGKWNDVPCSKQLLVICEF

SEQ ID NO: 63 - protein, 30 aa, Gallus gallus
LLTCDKPEEKMYSCPIIQCSAPAVNGLPGD

SEQ ID NO: 64 - protein, 301 aa, Bos taurus
EEMDVYSEKTLTDPCTLVVCAPPADSLRGHDGRDGKEGPQGEKGDPGPPGMPGPAGREGPSGRQGSMGPPGTPGPKGEPGPEGGVGAPGMPGSPGPAGLKGERGAPGPGGAIGPQGPSGAMGPPGLKGDRGDPGEKGARGETSVLEVDTLRQRMRNLEGEVQRLQNIVTQYRKAVLFPDGQAVGEKIFKTAGAVKSYSDAEQLCREAKGQLASPRSSAENEAVTQLVRAKNKHAYLSMNDISKEGKFTYPTGGSLDYSNWAPGEPNNRAKDEGPENCLEIYSDGNWNDIECREERLVICEF

SEQ ID NO: 65 - protein, 116 aa, Sus scrofa
AVGEKVFSTNGQSVAFDVIRELCARAGGRIAAPRSPEENEAIASIVKKHNTYAYLGLVEGPTAGDFFYLDGTPVNYTNWYPGEPRGRGKEKCVEMYTDGQWNDRNCQQYRLAICEF

SEQ ID NO: 66 - protein, 378 aa, Sus scrofa
MLLLPLSVLILLTQPPRSLGAEMKTYSQRAVANACALVMCSPMENGLPGRDGRDGREGPRGEKGDPGLPGAVGRAGMPGLAGPVGPKGDNGSTGEPGAKGDIGPCGPPGPPGIPGPAGKEGPSGQQGNIGPPGTPGPKGETGPKGEVGALGMQGSTGARGPAGLKGERGAPGERGAPGSAGAAGPAGATGPQGPSGARGPPGLKGDRGPPGERGAKGESGLPGITALRQQVETLQGQVQRLQKAFSQYKKVELFPNGRGVGEKIFKTGGFEKTFQDAQQVCTQAGGQMASPRSETENEALSQLVTAQNKAAFLSMTDIKTEGNFTYPTGEPLVYANWAPGEPNNNGGSSGAENCVEIFPNGKWNDKACGELRLVICEF

SEQ ID NO: 67 - protein, 244 aa, Mus musculus; identical to SEQ ID NO: 2

SEQ ID NO: 68 - protein, 239 aa, Mus musculus; identical to SEQ ID NO: 3

SEQ ID NO: 69 - protein, 301 aa, Bos taurus
EEMDVYSEKTLTDPCTLVVCAPPADSLRGHDGRDGKEGPQGEKGDPGPPGMPGPAGREGPSGRQGSMGPPGTPGPKGEPGPEGGVGAPGMPGSPGPAGLKGERGAPGPGGAIGPQGPSGAMGPPGLKGDRGDPGEKGARGETSVLEVDTLRQRMRNLEGEVQRLQNIVTQYRKAVLFPDGQAVGEKIFKTAGAVKSYSDAEQLCREAKGQLASPRSSAENEAVTQLVRAKNKHAYLSMNDISKEGKFTYPTGGSLDYSNWAPGEPGNRAKDEGPENCLEIYSDGNWNDIECREERLVICEF

SEQ ID NO: 70 - protein, 2412 aa, Homo sapiens [sequence omitted]

SEQ ID NO: 71 - protein, 277 aa, Homo sapiens; identical to SEQ ID NO: 56

SEQ ID NO: 72 - protein, 238 aa, Gallus gallus
MMATSLLTTDKPEEKMYSCPIIQCSAPAVNGLPGRDGRDGPKGEKGDPGEGLRGLQGLPGKAGPQGLKGEVGPQGEKGQKGERGIVVTDDLHRQITDLEAKIRVLEDDLSRYKKALSLKDVVNIGKKMFVSTGKKYNFEKGKSLCAKAGSVLASPRNEAENTALKDLIDPSSQAYIGISDAQTEGRFMYLSGGPLTYSNWKPGEPNNHKNEDCAVIEDSGKWNDLDCSNSNIFIICEL

SEQ ID NO: 73 - protein, 30 aa, Gallus gallus; identical to SEQ ID NO: 63

SEQ ID NO: 74 - protein, 60 aa, Bos taurus
AEMTTFSQKILANACTLVMCSPLESGLPGHDGQDGRECPHGEKGDPGSPGPAGRAGRPGW

SEQ ID NO: 75 - protein, 301 aa, Bos taurus
  MISC_FEATURE (7)..(): Unknown
EEMDVYXEKTLTDPCTLVVCAPPADSLRGHDGRDGKEGPQGEKGDPGPPGMPGPAGREGPSGRQGSMGPPGTPGPKGEPGPEGGVGAPGMPGSPGPAGLKGERGAPGPGGAIGPQGPSGAMGPPGLKGDRGDPGEKGARGETSVLEVDTLRQRMRNLEGEVQRLQNIVTQYRKAVLFPDGQAVGEKIFKTAGAVKSYSDAEQLCREAKGQLASPRSSAENEAVTQLVRAKNKHAYLSMNDISKEGKFTYPTGGSLDYSNWAPGEPGNRAKDEGPENCLEIYSDGNWNDIECREERLVICEF

SEQ ID NO: 76 - protein, 244 aa, Mus musculus; identical to SEQ ID NO: 2

SEQ ID NO: 77 - protein, 239 aa, Mus musculus; identical to SEQ ID NO: 3

SEQ ID NO: 78 - protein, 375 aa, Homo sapiens
MLLFLLSALVLLTQPLGYLEAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGALGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 79 - protein, 255 aa, Homo sapiens
MKALLALPLLLLLSTPPCAPQVSGIRGDALERFCLQQPLDCDDIYAQGYQSDGVYLIYPSGPSVPVPVFCDMTTEGGKWTVFQKRFNGSVSFFRGWNDYKLGFGRADGEYWLGLQNMHLLTLKQKYELRVDLEDFENNTAYAKYADFSISPNAVSAEEDGYTLFVAGFEDGGAGDSLSYHSGQKFSTFDRDQDLFVQNCAALSSGAFWFRSCHFANLNGFYLGGSHLSYANGINWAQWKGFYYSLKRTEMKIRRA

SEQ ID NO: 80 - protein, 15 aa, Homo sapiens
CRALIKRIQAMIPKG

SEQ ID NO: 81 - protein, 374 aa, Mus musculus
MLPFLSMLVLLVQPLGNLGAEMKSLSQRSVPNTCTLVMCSPTENGLPGRDGRDGREGPRGEKGDPGLPGPMGLSGLQGPTGPVGPKGENGSAGEPGPKGERGLSGPPGLPGIPGPAGKEGPSGKQGNIGPQGKPGPKGEAGPKGEVGAPGMQGSTGAKGSTGPKGERGAPGVQGAPGNAGAAGPAGPAGPQGAPGSRGPPGLKGDRGVPGDRGIKGESGLPDSAALRQQMEALKGKLQRLEVAFSHYQKAALFPDGRSVGDKIFRTADSEKPFEDAQEMCKQAGGQLASPRSATENAAIQQLITAHNKAAFLSMTDVGTEGKFTYPTGEPLVYSNWAPGEPNNNGGAENCVEIFTNGQWNDKACGEQRLVICEF

SEQ ID NO: 82 - protein, 248 aa, Canis familiaris
MWLRCLALALTLLMVSGIENNTKDVCVGNPGIPGTPGSHGLPGRDGRDGVKGDPGPPGPLGPPGGMPGHPGPNGMTGAPGVAGERGEKGEPGERGPPGLPASLDEELQTTLHDLRHQILQTMGVLSLHESLLVVGRKVFSSNAQSINFNDIQELCAGAGGQIAAPMSPEENEAVASIVKKYNTYAYLGLVESPDSGDFQYMDGAPVNYTNWYPGEPRGRGKEQCVEMYTDGQWNNKNCLQYRLAICEF

SEQ ID NO: 83 - protein, 247 aa, Oryctolagus cuniculus
MLLLSLALTLISAPASDTCDTKDVCIGSPGIPGTPGSHGLPGRDGRDGVKGDPGPPGPMGPPGGMPGLPGRDGLIGAPGVPGERGDKGEPGERGPPGLPAYLDEELQATLHELRHHALQSIGVLSLQGSMKAVGEKIFSTNGQSVNFDAIREVCARAGGRIAVPRSLEENEAIASIVKERNTYAYLGLAEGPTAGDFYYLDGDPVNYTNWYPGEPRGQGREKCVEMYTDGKWNDKNCLQYRLVICEF

SEQ ID NO: 84 - protein, 374 aa, Mus musculus; identical to SEQ ID NO: 81

SEQ ID NO: 85 - protein, 158 aa, Homo sapiens
EAEAGSVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 86 - protein, 158 aa, Homo sapiens; identical to SEQ ID NO: 85

SEQ ID NO: 87 - protein, 158 aa, Homo sapiens; identical to SEQ ID NO: 85

SEQ ID NO: 88 - protein, 2403 aa, Homo sapiens
MGISTVILEMCLLWGQVLSTGGWIPRTTDYASLIPSEVPLDTTVAEGSPFPSELTLESTAAEGSPISLESTLESTVAEGSLIPSESTLESTVAEGSDSGLALRLVNGDGRCQGRVEILYRGSWGTVCDDSWDTNDANVVCRQLGCGWAMSAPGNAWFGQGSGPIALDDVRCSGHESYLWSCPHNGWLSHNCGHGEDAGVICSAAQPQSTLRPESWPVRISPPVPTEGSESSLALRLVNGGDRCRGRVEVLYRGSWGTVCDDYWDTNDANVVCRQLGCGWAMSAPGNAQFGQGSGPIVLDDVRCSGHESYLWSCPHNGWLTHNCGHSEDAGVICSAPLSRPTPSPDTWPTSHASTAGPESSLALRLVNGGDRCQGRVEVLYRGSWGTVCDDSWDTSDANVVCRQLGCGWATSAPGNARFGQGSGPIVLDDVRCSGYESYLWSCPHNGWLSHNCQHSEDAGVICSDTLPTITLPASTVGSESSLALRLVNGGDRCQGRVEVLYRGSWGTVCDDSWDTNDANVVCRQLGCGWAMLAPGNARFGQGSGPIVLDDVRCSGNESYLWSCPHNGWLSHNCGHSEDAGVICSGPESSLALGLVNGGDRCQGRVEVLYRGSWGTVCDDSWDTNDANVVCRQLGCGWATSAPGNARFGQGSGPIVLDDVRCSGHESYLWSCPNNGWLSHNCGHHEDAGVICSAAQSRSTPRPDTLSTITLPPSTVGSESSLTLRLVNGSDRCQGRVEVLYRGSWGTVCDDSWDTNDANVVCRQLGCGWATSAPGNARFGQGSGPIVLDDVRCSGHESYLWSCPHNGWLSHNCGHHEDAGVICSVSQSRPTPSPDTWPTSHASTAGSESSLALRLVNGGDRCQGRVEVLYRGSWGTVCDDSWDTSDANVVCRRLGCGWATSAPGNARFGQGSGPIVLDDVRCSGYESYLWSCPHNGWLSHNCQHSEDAGVICSAAHSWSTPSPDTLPTITLPASTVGSESSLALRLVNGGDRCQGRVEVLYQGSWGTVCDDSWDTNDANVVCRQLGCGWAMSAPGNARFGQGSGPIVLDDVRCSGHESYLWSCPHNGWLSHNCGHSEDAGVICSASQSRPTPSPDTWPTSHASTAGSESSLALRLVNGGDRCQGRVEVLYRGSWGTVCDDYWDTNDANVVCRQLGCGWAMSAPGNARFGQGSGPIVLDDVRCSGHESYLWSCPHDGWLSHNCGHHEDAGVICSASQSQPTPSPDTWPTSHASTAGSESSLALRLVNGGDRCQGRVEVLYRGPWGTVCDDYWDTNDANVVCRQLGCGWATSAPGNARFGQGSGPIVLDDVRCSGHESYLWSCPHNGWLSHNCGHHEDAGVICSASQSQPTPSPDTWPTSHASTAGSESSLALRLVNGGDRCQGRVEVLYRGSWGTVCDDYWDTNDANVVCRQLGCGWATSAPGSARFGQGSGPIALDDVRCSGHESYLWSCPHNGWLSHNCGHHEDAGVICSASQSQPTPSPDTWPTSRASTAGSESTLALRLVNGGDRCRGRVEVLYQGSWGTVCDDYWDTNDANVVCRQLGCGWAMSAPGNAQFGQGSGPIVLDDVRCSGHESYLWSCPHNGWLSHNCGHHEDAGVICSAAQSQSTPRPDTWLTTNLPALTVGSESSLALRLVNGGDRCRGRVEVLYRGSWGTVCDDSWDTNDANVVCRQLGCGWAMSAPGNARFGQGSGPIVLDDVRCSGNESYLWSCPHKGWLTHNCGHHEDAGVICSATQINSTTTDWWHPTTTTTARPSSNCGGFLFYASGTFSSPSYPAYYPNNAKCVWEIEVNSGYRINLGFSNLKLEAHHNCSFDYVEIFDGSLNSSLLLGKICNDTRQIFTSSYNRMTIHFRSDISFQNTGFLAWYNSFPSDATLRLVNLNSSYGLCAGRVEIYHGGTWGTVCDDSWTIQEAEVVCRQLGCGRAVSALGNAYFGSGSGPITLDDVECSGTESTLWQCRNRGWFSHNCNHREDAGVICSGNHLSTPAPFLNITRPNTDYSCGGFLSQPSGDFSSPFYPGNYPNNAKCVWDIEVQNNYRVTVIFRDVQLEGGCNYDYIEVFDGPYRSSPLIARVCDGARGSFTSSSNFMSIRFISDHSITRRGFRAEYYSSPSNDSTNLLCLPNHMQASVSRSYLQSLGFSASDLVISTWNGYYECRPQITPNLVIFTIPYSGCGTFKQADNDTIDYSNFLTAAVSGGIIKRRTDLRIHVSCRMLQNTWVDTMYIANDTIHVANNTIQVEEVQYGNFDVNISFYTSSSFLYPVTSRPYYVDLNQDLYVQAEILHSDAVLTLFVDTCVASPYSNDFTSLTYDLIRSGCVRDDTYGPYSSPSLRIARFRFRAFHFLNRFPSVYLRCKMVVCRAYDPSSRCYRGCVLRSKRDVGSYQEKVDVVLGPIQLQTPPRREEEPR

SEQ ID NO: 89 - protein, 2413 aa, Homo sapiens
MGISTVILEMCLLWGQVLSTGGWIPRTTDYASLIPSEVPLDQTVAEGSPFPSESTLESTAAEGSPISLESTLESTVAEGSLIPSESTLESTVAEGSDSGLALRLVNGDGRCQGRVEILYRGSWGTVCDDSWDTNDANVVCRQLGCGWAMSAPGNAWFGQGSGPIALDDVRCSGHESYLWSCPHNGWLSHNCGHGEDAGVICSAAQPQSTLRPESWPVRISPPVPTEGSESSLALRLVNGGDRCRGRVEVLYRGSWGTVCDDYWDTNDANVVCRQLGCGWAMSAPGNAQFGQGSGPIVLDDVRCSGHESYLWSCPHNGWLTHNCGHSEDAGVICSAPQSRPTPSPDTWPTSHASTAGPESSLALRLVNGGDRCQGRVEVLYRGSWGTVCDDSWDTSDANVVCRQLGCGWATSAPGNARFGQGSGPIVLDDVRCSGYESYLWSCPHNGWLSHNCQHSEDAGVICSAAHSWSTPSPDTLPTITLPASTVGSESSLALRLVNGGDRCQGRVEVLYRGSWGTVCDDSWDTNDANVVCRQLGCGWAMLAPGNARFGQGSGPIVLDDVRCSGNESYLWSCPHNGWLSHNCGHSEDAGVICSGPESSLALRLVNGGDRCQGRVEVLYRGSWGTVCDDSWDTNDANVVCRQLGCGWAMSAPGNARFGQGSGPIVLDDVRCSGHESYLWSCPNNGWLSHNCGHHEDAGVICSAAQSRSTPRPDTLSTITLPPSTVGSESSLTLRLVNGSDRCQGRVEVLYRGSWGTVCDDSWDTNDANVVCRQLGCGWAMSAPGNARFGQGSGPIVLDDVRCSGHESYLWSCPHNGWLSHNCGHHEDAGVICSVSQSRPTPSPDTWPTSHASTAGSESSLALRLVNGGDRCQGRVEVLYRGSWGTVCDDSWDTSDANVVCRQLGCGWATSAPGNARFGQGSGPIVLDDVRCSGYESYLWSCPHNGWLSHNCQHSEDAGVICSAAHSWSTPSPDTLPTITLPASTVGSESSLALRLVNGGDRCQGRVEVLYQGSWGTVCDDSWDTNDANVVCRQPGCGWAMSAPGNARFGQGSGPIVLDDVRCSGHESYPWSCPHNGWLSHNCGHSEDAGVICSASQSRPTPSPDTWPTSHASTAGSESSLALRLVNGGDRCQGRVEVLYRGSWGTVCDDYWDTNDANVVCRQLGCGWAMSAPGNARFGQGSGPIVLDDVRCSGHESYLWSCPHNGWLSHNCGHHEDAGVICSASQSQPTPSPDTWPTSHASTAGSESSLALRLVNGGDRCQGRVEVLYRGSWGTVCDDYWDTNDANVVCRQLGCGWATSAPGNARFGQGSGPIVLDDVRCSGHESYLWSCPHNGWLSHNCGHHEDAGVICSASQSQPTPSPDTWPTSHASTAGSESSLALRLVNGGDRCQGRVEVLYRGSWGTVCDDYWDTNDANVVCRQLGCGWATSAPGNARFGQGSGPIVLDDVRCSGHESYLWSCPHNGWLSHNCGHHEDAGVICSASQSQPTPSPDTWPTSRASTAGSESTLALRLVNGGDRCRGRVEVLYQGSWGTVCDDYWDTNDANVVCRQLGCGWAMSAPGNAQFGQGSGPIVLDDVRCSGHESYLWSCPHNGWLSHNCGHHEDAGVICSAAQSQSTPRPDTWLTTNLPALTVGSESSLALRLVNGGDRCRGRVEVLYRGSWGTVCDDSWDTNDANVVCRQLGCGWAMSAPGNARFGQGSGPIVLDDVRCSGNESYLWSCPHKGWLTHNCGHHEDAGVICSATQINSTTTDWWHPTTTTTARPSSNCGGFLFYASGTFSSPSYPAYYPNNAKCVWEIEVNSGYRINLGFSNLKLEAHHNCSFDYVEIFDGSLNSSLLLGKICNDTRQIFTSSYNRMTIHFRSDISFQNTGFLAWYNSFPSDATLRLVNLNSSYGLCAGRVEIYHGGTWGTVCDDSWTIQEAEVVCRQLGCGRAVSALGNAYFGSGSGPITLDDVECSGTESTLWQCRNRGWFSHNCNHREDAGVICSGNHLSTPAPFLNITRPNTDYSCGGFLSQPSGDFSSPFYPGNYPNNAKCVWDIEVQNNYRVTVIFRDVQLEGGCNYDYIEVFDGPYRSSPLIARVCDGARGSFTSSSNFMSIRFISDHSITRRGFRAEYYSSPSNDSTNLLCLPNHMQASVSRSYLQSLGFSASDLVISTWNGYYECRPQITPNLVIFTIPYSGCGTFKQADNDTIDYSNFLTAAVSGGIIKRRTDLRIHVSCRMLQNTWVDTMYIANDTIHVANNTIQVEEVQYGNFDVNISFYTSSSFLYPVTSRPYYVDLNQDLYVQAEILHSDAVLTLFVDTCVASPYSNDFTSLTYDLIRSGCVRDDTYGPYSSPSLRIARFRFRAFHFLNRFPSVYLRCKMVVCRAYDPSSRCYRGCVLRSKRDVGSYQEKVDVVLGPIQLQTPPRREEEPR

SEQ ID NO: 90 - protein, 1785 aa, Homo sapiens
MGISTVILEMCLLWGQVLSTGGWIPRTTDYASLIPSEVPLDQTVAEGSPFPSESTLESTAAEGSPISLESTLESTVAEGSLIPSESTLESTVAEGSDSGLALRLVNGDGRCQGRVEILYRGSWGTVCDDSWDTNDANVVCRQLGCGWAMSAPGNAWFGQGSGPIALDDVRCSGHESYLWSCPHNGWLSHNCGHGEDAGVICSAAQPQSTLRPESWPVRISPPVPTEGSESSLALRLVNGGDRCRGRVEVLYRGSWGTVCDDYWDTNDANVVCRQLGCGWAMSAPGNAQFGQGSGPIVLDDVRCSGHESYLWSCPHNGWLTHNCGHSEDAGVICSAPQSRPTPSPDTWPTSHASTAGPESSLALRLVNGGDRCQGRVEVLYRGSWGTVCDDSWDTSDANVVCRQLGCGWATSAPGNARFGQGSGPIVLDDVRCSGYESYLWSCPHNGWLSHNCQHSEDAGVICSAAHSWSTPSPDTLPTITLPASTVGSESSLALRLVNGGDRCQGRVEVLYQGSWGTVCDDSWDTNDANVVCRQPGCGWAMSAPGNARFGQGSGPIVLDDVRCSGHESYPWSCPHNGWLSHNCGHSEDAGVICSASQSRPTPSPDTWPTSHASTAGSESSLALRLVNGGDRCQGRVEVLYRGSWGTVCDDYWDTNDANVVCRQLGCGWAMSAPGNARFGQGSGPIVLDDVRCSGHESYLWSCPHNGWLSHNCGHHEDAGVICSASQSQPTPSPDTWPTSHASTAGSESSLALRLVNGGDRCQGRVEVLYRGSWGTVCDDYWDTNDANVVCRQLGCGWATSAPGNARFGQGSGPIVLDDVRCSGHESYLWSCPHNGWLSHNCGHHEDAGVICSASQSQPTPSPDTWPTSRASTAGSESTLALRLVNGGDRCRGRVEVLYQGSWGTVCDDYWDTNDANVVCRQLGCGWAMSAPGNAQFGQGSGPIVLDDVRCSGHESYLWSCPHNGWLSHNCGHHEDAGVICSAAQSQSTPRPDTWLTTNLPALTVGSESSLALRLVNGGDRCRGRVEVLYRGSWGTVCDDSWDTNDANVVCRQLGCGWAMSAPGNARFGQGSGPIVLDDVRCSGNESYLWSCPHKGWLTHNCGHHEDAGVICSATQINSTTTDWWHPTTTTTARPSSNCGGFLFYASGTFSSPSYPAYYPNNAKCVWEIEVNSGYRINLGFSNLKLEAHHNCSFDYVEIFDGSLNSSLLLGKICNDTRQIFTSSYNRMTIHFRSDISFQNTGFLAWYNSFPSDATLRLVNLNSSYGLCAGRVEIYHGGTWGTVCDDSWTIQEAEVVCRQLGCGRAVSALGNAYFGSGSGPITLDDVECSGTESTLWQCRNRGWFSHNCNHREDAGVICSGNHLSTPAPFLNITRPNTDYSCGGFLSQPSGDFSSPFYPGNYPNNAKCVWDIEVQNNYRVTVIFRDVQLEGGCNYDYIEVFDGPYRSSPLIARVCDGARGSFTSSSNFMSIRFISDHSITRRGFRAEYYSSPSNDSTNLLCLPNHMQASVSRSYLQSLGFSASDLVISTWNGYYECRPQITPNLVIFTIPYSGCGTFKQADNDTIDYSNFLTAAVSGGIIKRRTDLRIHVSCRMLQNTWVDTMYIANDTIHVANNTIQVEEVQYGNFDVNISFYTSSSFLYPVTSRPYYVDLNQDLYVQAEILHSDAVLTLFVDTCVASPYSNDFTSLTYDLIRSGCVRDDTYGPYSSPSLRIARFRFRAFHFLNRFPSVYLRCKMVVCRAYDPSSRCYRGCVLRSKRDVGSYQEKVDVVLGPIQLQTPPRREEEPR

SEQ ID NO: 91 - protein, 34 aa, Bos taurus
LIPCCPVNIKRLLIVVVVVVLLVVVIVGALLMGL

SEQ ID NO: 92 - protein, 35 aa, Canis familiaris
LGIPCFPSSLKRLLIIVVVIVLVVVVIVGALLMGL

SEQ ID NO: 93 - protein, 371 aa, Bos taurus; identical to SEQ ID NO: 62

SEQ ID NO: 94 - protein, 375 aa, Homo sapiens; identical to SEQ ID NO: 78

SEQ ID NO: 95 - protein, 197 aa, Homo sapiens
MDVGSKEVLMESPPDYSAAPRGRFGIPCCPVHLKRLLIVVVVVVLIVVVIVGALLMGLHMSQKHTEMVLEMSIGAPEAQQRLALSEHLVTTATFSIGSTGLVVYDYQQLLIAYKPAPGTCCYIMKIAPESIPSLEALNRKVHNFQMECSLQAKPAVPTSKLGQAEGRDAGSAPSGGDPAFLGMAVNTLCGEVPLYYI

SEQ ID NO: 96 - protein, 248 aa, Canis familiaris
MWLRCLALALTLLMVSGIENNTKDVCVGNPGIPGTPGSHGLPGRDGRDGVKGDPGPPGPLGPPGGMPGHPGPNGMTGAPGVAGERGEKGEPGERGPPGLPASLDEELQTTLHDLRHQILQTMGVLSLHESLLVVGRKVFSSGAQSINFNDIQELCAGAGGQIAAPMSPEENEAVASIVKKYNTYAYLGLVESPDSGDFQYMDGAPVNYTNWYPGEPRGRGKEQCVEMYTDGQWNNKNCLQYRLAICEF

SEQ ID NO: 97 - protein, 248 aa, Homo sapiens [sequence omitted]

SEQ ID NO: 98 - protein, 301 aa, Bos taurus; identical to SEQ ID NO: 64

SEQ ID NO: 99 - protein, 369 aa, Bos taurus
MLLLPLSVLLLLTQPWRSLGAEMKIYSQKTMANACTLVMCSPPEDGLPGRDGRDGREGPRGEKGDPGSPGPAGRAGMPGPAGPIGLKGDNGSAGEPGPKGDTGPPGPPGMPGPAGREGPSGKQGSMGPPGTPGPKGDTGPKGGVGAPGIQGSPGPAGLKGERGAPGDPGAPGRAGAPGPRGAIGPQGPSGARGPPGLKGDRGTPGERGAKGESGLAEVNALRQRVGILEGQLQRLQNAFSQYKKAMLFPNGRSVGEKIFKTVGSEKTFQDAQQICTQAGGQLPSPRSGAENEALTQLATAQNKAAFLSMSDTRKEGTFIYPTGEPLVYSNWAPQEPNNDGGSENCVEIFPNGKWNDKVCGEQRLVICEF

SEQ ID NO: 100 - protein, 116 aa, Sus scrofa; identical to SEQ ID NO: 65

SEQ ID NO: 101 - protein, 378 aa, Sus scrofa; identical to SEQ ID NO: 66

SEQ ID NO: 102 - protein, 34 aa, Bos taurus; identical to SEQ ID NO: 91

SEQ ID NO: 103 - protein, 369 aa, Bos taurus
MLLLPLSVLLLLTQPWRSLGAEMKIYSQKTMANACTLVMCSPPEDGLPGRDGRDGREGPRGEKGDPGSPGPAGRAGMPGPAGPIGLKGDNGSAGEPGPKGDTGPPGPPGMPGPAGREGPSGKQGSMGPPGTPGPKGDTGPKGGVGAPGIQGSPGPAGLKGERGAPGEPGAPGRAGAPGPAGAIGPQGPSGARGPPGLKGDRGTPGERGAKGESGLAEVNALRQRVGILEGQLQRLQNAFSQYKKAMLFPNGRSVGEKIFKTVGSEKTFQDAQQICTQAGGQLPSPRSGAENEALTQLATAQNKAAFLSMSDTRKEGTFIYPTGEPLVYSNWAPQEPNNDGGSENCVEIFPNGKWNDKVCGEQRLVICEF

SEQ ID NO: 104 - protein, 301 aa, Bos taurus; identical to SEQ ID NO: 69

SEQ ID NO: 105 - protein, 2412 aa, Homo sapiens [sequence omitted]

SEQ ID NO: 106 - protein, 2413 aa, Homo sapiens; identical to SEQ ID NO: 89

SEQ ID NO: 107 - protein, 374 aa, Mus musculus; identical to SEQ ID NO: 81

SEQ ID NO: 108 - protein, 35 aa, Canis familiaris; identical to SEQ ID NO: 92

SEQ ID NO: 109 - protein, 34 aa, Bos taurus; identical to SEQ ID NO: 91

SEQ ID NO: 110 - protein, 35 aa, Homo sapiens
GQSITFDAGKEQCVEMYTDGQWNDRNCLYLTICEF

SEQ ID NO: 111 - protein, 370 aa, Oryctolagus cuniculus
MAKSHLPPWLLLLLLPTLCGPGTAVWATSPLACAQGPEFWCQSLEQALQCKALGHCLQEVWGHVGADDLCQECQDIVNILTKMTKEAIFQDTIRKFLEHECDVLPLKLLVPQCHHVLDVYFPLTITYFLSQINAKAICQHLGLCQPGSPEPPLDPLPDKLVLPTLLGALPAKPGPHTQDLSAQRFPIPLPLCWLCRTLLKRIQAMIPKGVLAMAVAQVCHVVPLVVGGICQCLAERYTVILLEVLLGHVLPQLVCGLVLRCSSVDSIGQVPPTLEALPGEWLPQDPECPLCMSVTTQARNISEQTRPQAVYHACLSSQLDKQECEQFVELHTPQLLSLLSRGWDARAICQALGACVATLSPLQCIQSPHF

SEQ ID NO: 112 - protein, 247 aa, Oryctolagus cuniculus
MLLLSLALTLISAPASDTCDTKDVCIGSPGIPGTPGSHGLPGRDGRDGVKGDPGPPGPMGPPGGMPGLPGRDGLIGAPGVPGERGDKGEPGERGPPGLPAYLDEELQATLHELRHHALQSIGVLSLQGSMKAVGEKIFSTNGQSVNFDAIREVCARAGGRIAIVKEVPRSLEENEAIASRNTYAYLGLAEGPTAGDFYYLDGDPVNYTNWYPGEPRGQGREKCVEMYTDGKWNDKNCLQYRLVICEF

SEQ ID NO: 113 - protein, 369 aa, Bos taurus; identical to SEQ ID NO: 103

SEQ ID NO: 114 - protein, 301 aa, Bos taurus; identical to SEQ ID NO: 75
  MISC_FEATURE (7)..(7): Unknown

SEQ ID NO: 115 - protein, 375 aa, Homo sapiens
MLLFLLSALVLLTQPLGYLEAEMKTYSHRTTPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNAGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 116 - protein, 194 aa, Rattus norvegicus
MDMGSKEVLMESPPDYSTGPRSQFRIPCCPVHLKRLLIVVVVVVLVVVVIVGALLMGLHMSQKHTEMVLEMSIGGAPETQKRLALSEHTDTIATFSIGSTGIVLYDYQRLLTAYKPAPGTYCYIMKMAPESIPSLEALARKFKNFQAKSSTPTSKLGQEEGHSAGSDSDSSGRDLAFLGLAVSTLCGVLPLYYI

SEQ ID NO: 117 - protein, 247 aa, Oryctolagus cuniculus
MLLLSLALTLISAPASDTCDTKDVCIGSPGIPGTPGSHGLPGRDGRDGVKGDPGPPAPWAPPGGMPGLPGRDGLIGAPGVPGERGDKGEPGERGPPGLPAYLDEELQATLHELRHHALQSIGVLSLQGSMKAVGEKIFSTNGQSVNFDAIREVCARAGGRIAVPRSLEENEAIASIVKERNTYAYLGLAEGPTAGDFYYLDGDPVNYTNWYPGEPRGQGREKCVEMYTDGKWNDKNCLQYRLVICEF

SEQ ID NO: 118 - protein, 251 aa, Homo sapiens
  MISC_FEATURE (25)..(130): Polypeptide sequence from ficolin dom aa131
MELDRAVGVLGAATLLLSFLGMAWALQAADTCPEVKMVGLEGSDKLTILRGCPGLPGAPGDKGEAGTNGKRGERGPPGPPGKAGPPGPNGAPGEPQPCLTGPRTCKDLLDRGHFLSGWHTIYLPDCRPLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 119 - protein, 329 aa, Homo sapiens
  MISC_FEATURE (25)..(206): Polypeptide sequence from ficolin-dom. containing pred. coil-coil to aa207
MELDRAVGVLGAATLLLSFLGMAWALQAADTCPEVKMVGLEGSDKLTILRGCPGLPGAPGDKGEAGTNGKRGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 120 - protein, 240 aa, Homo sapiens
  MISC_FEATURE (25)..(91): Polypeptide sequence from collagen
MELDRAVGVLGAATLLLSFLGMAWALQAADTCPEVKMVGLEGSDKLTILRGCPGLPGAPGDKGEAGTNGKRGERGPPGPPGKAGPPGPNGAPPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 121 - protein, 255 aa, Homo sapiens
  MISC_FEATURE (25)..(82): Polypeptide sequence from collagen to cons.K at aa 93
MELDRAVGVLGAATLLLSFLGMAWALQAADTCPEVKMVGLEGSDKLTILRGCPGLPGAPGDKGEAGTNGKRGERGPPGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 122 - protein, 254 aa, Homo sapiens
  MISC_FEATURE (25)..(69): Polypeptide sequence from collagen
MELDRAVGVLGAATLLLSFLGMAWALQAADTCPEVKMVGLEGSDKLTILRGCPGLPGAPGDKGEAGTNGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 123 - protein, 232 aa, Homo sapiens
  MISC_FEATURE (21)..(41): Polypeptide sequence from MBL collagen
MSLFPSLPLLLLSMVAASYSETVTCEDAQKTCPAVIACSSPGCPGLPGAPGDKGEAGTNGKRGERGPPGPPGKAGPPGPNGAPSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 124 - protein, 237 aa, Homo sapiens
  MISC_FEATURE (21)..(89): Polypeptide sequence from collagen
MSLFPSLPLLLLSMVAASYSALQAADTCPEVKMVGLEGSDKLTILRGCPGLPGAPGDKGEAGTNGKRGERGPPGPPGKAGPPGPNGAPSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 125 - protein, 288 aa, Homo sapiens
LQAADTCPEVKMVGLEGSDKLTILRGCPGLPGAPGDKGEAGTNGKRGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 126 - protein, 228 aa, Homo sapiens
ETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 127 - protein, 216 aa, Homo sapiens
LQAADTCPEVKMVGLEGSDKLTILRGCPGLPGAPGDKGEAGTNGKRGERGPPGPPGKAGPPGPNGAPSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

(19) United States
(12) Patent Application Publication
(10) Pub. No.: US 2006/0188963 A1
(43) Pub. Date: Aug. 24, 2006

(54) Collectin-complement activating protein chimeras

(75) Inventors: Leif Kongerslev, Birkerod (DK); Dietmar Weilguny, Virum (DK); Finn Matthiesen, Bronshoj (DK)

Correspondence Address: BROWDY AND NEIMARK, P.L.L.C.;624 NINTH STREET, NW, SUITE 300, WASHINGTON, DC, 20001-5303, US

(73) Assignee: Natlmmune A/S, Kobenhavn O (DK)

(21) Appl. No.: 10/527,191

(22) PCT Filed: Sep. 10, 2003
(86) PCT No.: PCT/DK03/00585

§ 371 Date: Mar. 10, 2005

(30) Foreign Application Priority Data
Sep. 10, 2002 (DK) PA 2002 01328

(51) Int. Cl. C07K 14/74; C07H 21/04; C12P 21/04

(52) U.S. Cl. 435/69.7; 435/320.1; 435/325; 530/396; 536/23.5

(57) ABSTRACT

The present invention relates to a fusion protein capable of activating the complement system, the fusion protein comprising a first polypeptide sequence derived from a lectin-complement pathway activating protein or a functional homologue thereof; and a second polypeptide sequence derived from a collectin or a functional homologue thereof; wherein said complement activating protein is not a collectin. A preferred fusion protein comprises amino acids of the L-ficolin sequence of FIG. 1 and amino acids of the MBL sequence shown in FIG. 2. The fusion protein is suitable for use in treatment consisting of creation, reconstitution, enhancing and/or stimulating the opsonic and/or bactericidal activity of the complement system, i.e. enhancing the ability of the immune defence to recognise and kill microbial pathogens, and accordingly, the invention relates to a medicament comprising the fusion protein, methods for producing said fusion protein and methods for treating diseases, in particular infections.

### FIELD OF INVENTION

The present invention relates to a fusion protein capable of activating the complement system, methods for producing said fusion protein as well as pharmaceutical composition comprising said fusion protein and methods for treating diseases, in particular infections, with said fusion protein.

### BACKGROUND OF INVENTION

Animals have developed different complex strategies to protect themselves against infections. The immune responses can be divided into to main groups, the adaptive immune response, in which an adaptation has taken place and in which cells play a dominant part and the innate immune response, which is available instantly and which primarily is based on molecules present in the body fluids. The innate immune system is operational at time of birth, in contrast to the adaptive immune defence which only during infancy, obtains its full power of protecting the body (Janeway et al., 1999).

Bacteria entering the body at mucosal surfaces or through broken skin are immediately recognised by collectins, a family of soluble proteins that recognise distinctive carbohydrate configurations that are present on the surfaces of microbes and absent from the cells of the multicellular organism. Collectins thus belong to the large and diverse group of pattern recognition receptors of the innate immune system. In humans, three collectins are known, although others may exist: cows for example have more. Collectins target the particles to which they bind either for uptake by phagocytes or for activation of the complement cascade, and in these ways can mediate their destruction.

Collectins all exhibit the following architecture: they have an N-terminal cysteine-rich region that appears to form inter-chain disulfide bonds, followed by a collagen-like region, an α-helical coiled-coil region and finally a C-type lectin domain which is the pattern-recognizing region and is referred to as the carbohydrate recognition domain (CRD). The name collectin is derived from the presence of both collagen- and lectin domains. The α-helical coiled-coil region initiates trimerisation of the individual polypetides to form collagen triple coils, thereby generating collectin subunits each consisting of 3 individual polypeptides, whereas the N-terminal region mediates form ation of oligomers of subunits. Different collectins exhibit distinctive higher order structures, typically either tetramers of subunits or hexamers of subunits. The grouping of large numbers of binding domains allows collectins to bind with high avidity to microbial cell walls, despite a relatively low intrinsic affinity of each individual CRD for carbohydrates.

C-type CRDs are found in proteins with a widespread occurrence, both in phylogenetic and functional perspective. The different CRDs of the different collectins enable them to recognise a range of distinct microbial surface components exposed on different microorganisms. The terminal CRDs are distributed in such a way that all three domain target surfaces that present binding sites has a spacing of approximately 53 Å (Sheriff et al., 1994; Weis & Drickamer, 1994). This property of ‘pattern recognition’ may contribute further to the selectively binding of microbial surfaces. The collagenous region or possibly the N-terminal tails of the collectins, are recognised by specific receptors on phagocytes; and is the binding site for associated proteases that are activated to initiate the complement cascade upon binding of the CRD domain to a target.

Mannan-binding lectin (MBL) also termed mannose-binding lectin or mannose binding protein is a collectin which has gained great interest as an important part of the innate immune system. MBL binds to specific carbohydrate structures found on the surface of a range of microorganisms including bacteria, yeast, parasitic protozoa and viruses, and has been found to exhibit antibacterial activity through killing mediated by activation of the terminal, lytic complement components or through promotion of phagocytosis. MBL deficiency its associated with susceptibility to frequent infections by a variety of microorganisms in childhood, and possibly also in adults.

The CRD of MBL recognises preferentially hexoses with equatorial 3- and 4-OH groups, such as mannose, glucose, N-acetylmannosamin and N-acetyl glucoseamin while carbohydrates which do not fulfil this sterical requirement, such as galactose and D-fucose, are not bound (Weis et al., 1992). The carbohydrate selectivity is obviously an important aspect of the self/non-self discrimination by MBL and is probably mediated by the difference in prevalence of mannose and N-acetyl glucoseamin residues on microbial surfaces, one example being the high content of mannose in the cell wall of yeasts such as Saccharomyces cerevisiae and Candida albicans. Carbohydrate structures in glycosylation of mammalian proteins are usually completed with sialic acid, which prevents binding of MBL to these oligomeric carbohydrates and thus prevents MBL recognition of ‘self’ surfaces. Also, the trimeric structure of each MBL subunit may be of importance for target recognition.

Complement is a group of proteins present in blood plasma and tissue fluid that aids the body's defences following an infection. The complement system is being activated through at least three distinct pathways, designated the classical pathway, the alternative pathway, and the MBLectn pathway (Janeway et al., 1-999). The classical pathway is initiated when complement factor 1 (C1) recognises surface-bound immunoglobulin. The C1 complex is composed of two proteolytic enzymes, C1r and C1s, and a non-enzymatic part, C1q, which contains immunoglobulin-recognising domains. C1q and MBL shares structural features, both molecules having a bouquet-like appearance when visualised by electron microscopy. Also, like C1q, MBL is found in complex with two proteolytic enzymes, the mannan-binding lectin associated proteases (MASP). The three pathways all generate complement factor 3 (C3) convertase, which ensures the binding of C3b to the surface of the activating surface, i.e. the targeted microbial pathogen. Conversion of C3 into surface bound C3b is pivotal in the process of eliminating the microbial pathogen by phagocytosis or lysis (Janeway et al., 1999).

Certain O-antigen specific oligosaccharides of Salmonella have been reported to activate complement in C4-deficient guinea-pig serum and Salmonella serogroup C was later shown to react with MBL and hence activate complement by the MBLectin pathway, which is also termed the MBL pathway of complement activation or the lectin pathway.

It has for some time-been speculated that the innate immune system may collaborate with the adaptive immune system in the generation of specific immune responses as exemplified by the antibody response after infection or vaccination. Fearon's group have shown that the attachment of the C3d fragment of complement factor C3 onto a protein antigen through fusion by gene technology can in crease the immunogenecity of the antigen 1000 fold or more. Practical applications of this technique, or any number of modifications, are still awaited.

The importance of the complement system for normal immune responses was first suggested by Pepys, who found impaired antibody responses to sheep erythrocytes, a thymus-dependent antigen, in mice that were C3-depleted with cobra venom factor. The idea of a link between innate and adaptive immunity was supported by reports demonstrating reduced primary antibody responses to thymus dependent antigens and impaired IgM to IgG switching in patients and experimental animals with deficiencies of C4, C2 and C3. The mechanism may involve the generation of C3-derived ligands for binding of antigen or antigen-containing complexes to complement receptors on B lymphocytes or antigen-presenting cells. Thus, blocking of CR1 (CD35) and CR2 (CD21) in mice with specific anti CR1 and anti-CR2 antibodies or with soluble receptor protein reduced antibody responses to immunisation and experiments with CR1 and CR2-deficient knock-out mice show the requirement of these receptors for responses to thymus-dependent antigens. In addition, patients with leucocyte adhesion deficiency, who lack the CD11/CD18 adhesion molecule CR3, demonstrate impaired antibody responses and failure to switch from IgM to IgG. The C3-derived fragment C3d, a specific CR2 ligand, as mentioned above, show a strong dose-dependent adjuvant effect.

Deficiencies of the classical complement pathway (C1, C2, C4 and C3) are associated with infections by encapsulated bacteria. The main reason for this is probably the reduced efficiency of opsonic and bactericidal defence mechanisms caused by complement dysfunction. However, impaired immune responses to polysaccharide antigens might also be considered. The influence of complement on responses to thymus-independent antigens has not been extensively studied, and the available information is contradictory. Thus, low antibody responses to thymus-independent antigens have been clearly documented in C3-depleted mice and C3-deficient dogs. On the other hand, some reports find that C3-deficient patients appear to respond normally to immunisation with polysaccharide vaccines.

Ficolins, like MBL, are lectins that contain a collagen-like domain. Unlike MBL, however, they have a fibrinogen-like domain, which is similar to fibrinogen β- and γ-chains. Ficolins also forms oligomers of structural subunits, each of which is composed of three identical 35 kDa polypeptides. Each subunit is composed of an amino-terminal, cysteine-rich region; a collagen-like domain that consists of tandem repeats of Gly-Xaa-Yaa triplet sequences (where Xaa and Yaa represent any amino acid); a neck region; and a fibrinogen-like domain. The oligomers of ficolins comprises two or more subunits, especially a tetrameric form of ficolin has been observed.

Some of the ficolins triggers the activation of the complement system substantially in similar way as done by MBL. This triggering of the complement system results in the activation of novel serine proteases (MASPs) as described above.

The fibrinogen-like domain of several lectins has a similar function to the CRD of C-type lectins including MBL, and hereby function as pattern-recognition receptors to discriminate pathogens from self.

Serum ficolins have a common binding specificity for GlcNAc (N-acetyl-glucosamine), elastin or GalNAc (N-acetyl-galactosamine). The fibrinogen-like domain is responsible for the carbohydrate binding. In human serum, two types of ficolin, known as L-ficolin (P35, ficolin L, ficolin 2 or hucolin) and H-ficolin (Hakata antigen, ficolin 3 or thermolabile b2-macroglycoprotein), have been identified, and both of them have lectin activity. L-ficolin recognises GlcNAc and H-ficolin recognises GalNAc. Another ficolin known as M-ficolin (P35-related protein, Ficolin 1 or Ficolin A) is not considered to be a serum protein and is found in leucocytes and in the lungs. L-ficolin and H-ficolin activate the lectin-complement pathway in association with MASPs. M-Ficolin, L-ficolin and H-ficolin has calcium-independent lectin activity.

MASPs (MBL-associated serine proteases) comprising MASP-1, MASP-2 and MASP-3 are proteolytic enzymes that are responsible for activation of the lectin pathway. The overall structure of MASPs resembles that of the two proteolytic components of the first factor in the classical complement pathway, C1r and C1s. The lectin pathway is initiated when MBL or a ficolin associated with MASP-1, MASP-2, MASP-3 and sMAP binds to a carbohydrate structure of the surfaces of e.g. bacteria, yeast, parasitic protoxoa, viruses. MASP-2 is the enzyme component that—like C1s in the classical pathway—cleaves the complement components C4 and C2 to form the C3 convertase C4bC2a, which is common to both the lectin- and classical-pathway activation routes.

MASP-1, MASP-2, MASP-3 and sMAP are encoded by two genes; sMAP is a truncated form of MASP-2, and MASP-3 is produced from the MASP-1 gene by alliterative splicing. The MASP-1 gene has an H-chain-encoding region that is common to MASP-1 and MASP-3, which is followed by tandem repeats of protease-domain-encoding regions that are specific to MASP-3 and MASP-1.

The MASP family can be divided into two phylogenetic lineages—TCN-type and AGY-type lineages. The TCN-type lineage, which includes MASP-1, has a TCN codon (where N denotes A, G, C or T) that encodes the active-site serine, the presence of a histidine-loop disulphide bridge and split exons. By contrast, the AGY-type lineage, which includes MASP-2, MASP-3, C1r and C1s, is characterised by an AGY codon (where Y denotes C or T) that encodes the active-site serine, the absence of a histidine-loop and a single exon.

MASP-1, MASP-2, MASP-3, C1r and C1s consist or six domains: two C1r/C1s/Uegf/bone morphogenetic protein 1 (CUB) domains; an epidermal growth factor (EGF)-like domain; two complement control protein (CCP) domains or short consensus repeats (SCRs), and a serine-protease domain. Histidine (H), aspartic acid (D) and serine (S) residues are essential for the formation of the active centre in the serine-protease domain. Only MASP-1 has two additional cysteine residues in a light chain, which form a histidine.loop disulphide bridge (S—S), as is found in trypsin and chymotrypsin. On binding of MBL and ficolins to carbohydrate on the surface of a pathogen, the pro-enzyme form of a MASP is cleaved between the second CCP and the protease domain, which results in an active form that consists of two polypeptides—heavy and light chains (also known as A and B chains).

### SUMMARY OF INVENTION

The present invention relates to fusion proteins capable of activating the complement system. Accordingly, the present invention relates to a fusion protein comprising a first polypeptide sequence derived from a lectin-complement pathway activating protein (=complement activating protein) or a functional homologue thereof; and a second polypeptide sequence derived from a collectin or a functional homologue thereof;

wherein said complement activating protein is not a collectin.

The fusion protein is suitable for use in treatment consisting of creation, reconstitution, enhancing and/or stimulating the opsonic and/or bactericidal activity of the complement system, i.e. enhancing the ability of the immune defence to recognise and kill microbial pathogens, and accordingly, the invention relates to a medicament comprising the fusion protein.

Also, in another aspect the invention relates to a method of treatment of a clinical condition in an individual in need thereof comprising administering to said individual the fusion protein as defined above.

In another aspect the invention relates to a method of treatment or prophylaxis of a clinical condition, such as infection, in an individual in need thereof comprising administering to said individual a the fusion protein a first polypeptide sequence derived from a protein capable of forming oligomers of structural units; and a second polypeptide sequence derived from a mannose binding lectin (MBL, wherein said first polypeptide sequence and said second peptide sequence is not derived from the same protein, and said fusion protein is capable of associating with mannose-associated serine protease (MASP). The first polypeptide sequence is preferably derived from a protein capable of forming tetramers, pentamers, and/or hexamers of a structural unit. In a preferred embodiment the first polypeptide sequence and the second polypeptide sequence are as described below.

In a further aspect the invention relates to use of the fusion protein as defined above for the preparation of a medicament for the treatment of a clinical condition in an individual in need thereof.

Furthermore the invention relates to a method for producing the fusion protein, as well as an isolated nucleic acid sequence encoding the fusion protein, a vector comprising the sequence and a cell comprising the vector.

### DRAWINGS

FIG. 1 shows the sequence of L ficolin

FIG. 2 shows the sequence of MBL

FIG. 3 shows an example of a fusion protein

FIGS. 4-8 show plasmids as described in Example 1

FIG. 9 shows an alignment of fusion proteins described in Example 1

FIG. 10 shows two Western blots as discussed in Example 2.

### DEFINITIONS

Collectins: A family of structurally related, carbohydrate-recognising proteins of innate immunity, including mannan-binding lectin (MBL) and surfactant proteins A and D. The name refers to the presence of a collagen-like region and a C-type lectin domain.

Complement: A group of proteins present in blood plasma and tissue fluid that aids the body's defences following an infection. Complement is involved in destroying foreign cells and attracting phagocytes to the area of conflict in the body.

Conjugated: An association formed between two compounds for example between an immunogenic determinant and a collectin and/or collectin homologue or between an immunogenic determinant and a saccharide. The association may be a physical association generated e.g. by the formation of a chemical bond, such as e.g. a covalent bond.

CRD: Carbohydrate recognition domain, a C-type lectin domain that is found at the C-terminus of collectins.

Immunogenic determinant: A molecule, or a part thereof, containing one or more epitopes that will stimulate the immune system of a host organism to make a secretory, humoral and/or cellular antigen-specific response, or a DNA molecule which is capable of producing such an immunogen in a vertebrate.

Immune response: Response to a immunogenic composition comprising an immunogenic determinant. An immune response involves the development in the host of a cellular- and/or humoral immune response to the administered composition or vaccine in question. An immune response generally involves the action of one or more of i) the antibodies raised, ii) B cells, iii) helper T cells, iv) suppressor T cells, v) cytotoxic T cells and iv) complement directed specifically or unspecifically to an immunogenic determinant present in an administered immunogenic composition.

Lectin: Proteins that specifically bind carbohydrates.

MASP: Mannose-associated serine protease

MBL: Mannan-binding lectin or mannose-binding lectin.

Subunit complex=structural unit: complex of three individual fusion proteins, like the subunit complex discussed above for MBL and ficolins.

### DETAILED DESCRIPTION OF THE INVENTION

An object of the present invention is to provide a fusion protein capable of activating the complement system in order to aid in preventing or treating diseases, in particular infectious diseases.

The fusion protein is composed of

a first polypeptide sequence derived from a lectin-complement pathway activating protein (=complement activating protein) or from a functional homologue thereof; and

a second polypeptide sequence derived from a collectin or from a functional homologue thereof;

wherein said complement activating protein is not a collectin.

By combining a polypeptide sequence derived from a lectin-complement pathway activating protein and a polypeptide sequence derived from a collectin it is possible to design a fusion protein having binding affinity for a variety of carbohydrates, preferably bacterial and viral carbohydrates and at the same time having complement system activating activity.

First Polypeptide Sequence

The first polypeptide sequence may be derived from any lectin-complement pathway activating protein. Said lectin-complement pathway activating protein may be naturally occurring lectin-complement pathway activating protein as well as variants or homologues to said lectin-complement pathway activating proteins, wherein said variants or homologues have maintained the lectin-complement pathway activating activity.

It is preferred that the fusion protein is capable of forming subunit complexes, each consisting of 3 individual fusion proteins as defined above.

Also the first polypeptide sequence is preferably capable of forming oligomeric complexes with the first polypeptide sequence of another fusion protein, wherein said another fusion protein may be identical to the first fusion protein. Thereby an oligomeric complex of two or more fusion proteins or two or more subunit complexes may be provided, said oligomeric complex having a higher binding avidity for bacterial or viral carbohydrates than the monomeric fusion protein. In a preferred embodiment the oligomeric complex is a dimeric subunit complex, more preferably a trimeric subunit complex, more preferably a tetrameric subunit complex.

In a preferred embodiment the lectin-complement pathway activating protein is a ficolin as defined above. Said ficolin may be L-ficolin, H-ficolin or M-ficolin or variants or homologues thereof. In a preferred embodiment the lectin-complement pathway activating protein is L-ficolin.

In another embodiment the first polypeptide sequence comprises the fibrinogen domain of the lectin, and/or the neck region of a lectin, such as a ficolin or a homologue or a variant thereof.

When the first polypeptide sequence is derived from ficolin or from a variant or a homologue of ficolin, it is preferred that the first polypeptide sequence comprises the collagen-like domain from ficolin or from a variant or homologue of ficolin. In another embodiment it is preferred that the first polypeptide sequence comprises the Cystein rich domain from ficolin or from a variant or homologue of ficolin. It is even more preferred that the first polypeptide sequence comprises the collagen-like domain and the Cystein rich domain from ficolin or from a variant or homologue of ficolin.

It is more preferred that the first polypeptide sequence comprises the collagen-like domain from L-ficolin or from a variant or homologue of L-ficolin. In another embodiment it is more preferred that the first polypeptide sequence comprises the Cystein rich domain from L-ficolin or from a variant or homologue of L-ficolin. It is even more preferred that the first polypeptide sequence comprises the N-terminal region of L-ficolin including two Cystein amino acid residues.

It is even more preferred that the first polypeptide sequence comprises the collagen-like domain and the Cystein rich domain from L-ficolin or from a variant or homologue of L-ficolin.

In a particular preferred embodiment the ficolin has one of the sequences listed below with reference to their database and accession No. For each of the sequences the Cystein rich region and the collagen-like region is described.

NP_{—}003656. ficolin 3 precursor; ficolin (collagen/fibrinogen domain-containing) 3 (Hakata antigen) [Homo sapiens] [gi:4504331]

90 . . . 299/region_name=“pfam00147, fibrinogen_C, Fibrinogen beta and gamma chains, C-terminal globular domain”

90 . . . 299/region_name=“smart00186, FBG, Fibrinogen-related domains (FReDs);

Domain present at the C-termini of fibrinogen beta and gamma chains, and a variety of fibrinogen-related proteins, including tenascin and Drosophila scabrous”

1 mdllwilpsl wllllggpac lktqehpscp gpreleaskv vllpscpgap gspgekgapg

61 pqgppgppgk mgpkgepgdp vnllrcqegp rncrellsqg atlsgwyhlc lpegralpvf

121 cdmdtegggw lvfqrrqdgs vdffrswssy ragfgnqese fwlgnenlhq ltlqgnwelr

181 veledfngnr ffahyaffrl lgevdhyqla lgkfsegtag dslslhsgrp fttydadhds

241 snsncavivh gawwyascyr snlngryavs daaahkygid wasgrgvghp yrrvrmmlr

XP_{—}116792. similar to Ficolin 2 precursor (Collagen/fibrinogen domain-containing protein 2) (Ficolin-B) (Ficolin B) (Serum lectin P35) (EBP-37) (Hucolin) (L-Ficolin) [Homo sapiens] [gi:20477458]

91 . . . 168/region-name=“pfam00147, fibrinogen_C, Fibrinogen beta and gamma chains, C-terminal globular domain”

91 . . . 168/region-name=“smart00186, FBG, Fibrinogen-related domains (FReDs);

Domain present at the C-termini of fibrinogen beta and gamma chains, and a variety of fibrinogen-related proteins, including tenascin and Drosophila scabrous”

1 mgpallalsf lwtmaltedt cpamleyval nsepgmaskn psrrhglsll vvdqgpgarg

61 vrtdqgpsga dpgslelhge cpifpseqvi lthhnnypfs tedqdndrda encavhyqga

121 wwyaschish lngvylggar dsftnginwk sgkgnnysyk vsemkvrpt

O00602. Ficolin 1 precursor (Collagen/fibrinogen domain-containing protein 1) (Ficolin-A) (Ficolin A) (M-Ficolin) [gi:20455484]

1 . . . 29/gene=“FCN1”/region_name=“Signal”/note=“POTENTIAL.”

30 . . . 326/gene=“FCN1”/region_name=“Mature chain”/note=“FICOLIN 1.”

55 . . . 93/gene=“FCN1”/region_name=“Domain”/note=“COLLAGEN-LIKE.”

133/gene=“FCN1”/region-name=“Conflict”/note=“T->N (IN REF. 1).”

144 . . . 290/gene=“FCN1”/region-name=“Domain”/note=“FIBRINOGEN C-TERMINAL.”

287/gene=“FCN1”/region-name=“Conflict”/note=“N->S (IN REF. 1).”

305/gene=“FCN1”/sitetype=“glycosylation”/note=“N-LINKED (GLCNAC . . . ) (POTENTIAL).”

1 melsgatmar glavllylfl hiknipaqaa dtcpevkwg legsdkltil rgcpglpgap

61 gpkgeagvig ergerglpga pgkagpvgpk gdrgekgmrg ekgdagqsqs catgprnckd

121 lldrgyflsg whtiylpdcr pltylcdmdt dgggwtvfqr rmdgsvdfyr dwaaykqgfg

181 sqlgefwlgn dnihaltaqg sselrvdlvd fegnhqfaky ksfkvadeae kyklvigafv

241 ggsagnsitg hnnnffstkd qdndvsssnc aekfqgawwy adchasnlng lylmgphesy

301 anginwsaak gykysykvse mkvrpa//

O75636. Ficolin 3 precursor (Collagen/fibrinogen domain-containing protein 3) (Collagen/fibrinogen domain-containing lectin 3 P35) (Hakata antigen) [gi-13124185]

1 . . . 21/gene=“FCN3”/region_name=“Signal”/note=“POTENTIAL.”

22 . . . 299/gene=“FCN3”/region_name=“Mature chain”/note=“FICOLIN 3.”

48 . . . 80/gene=“FCN3”/region_name=“Domain”/note=“COLLAGEN-LIKE.”

50/gene=“FCN3”/sitetype=“hydroxylation”

53/gene=“FCN3”/sitetype=“hydroxylation”

59/gene=“FCN3”/site_type=“hydroxylation”

65/gene=“FCN3”/site_type=“hydroxylation”

68/gene=“FCN3”/sitetype=“hydroxylation”

77/gene=“FCN3”/site_type=“hydroxylation”

119 . . . 265/gene=“FCN3”/region_name=“Domain”/note=“FIBRINOGEN C-TERMINAL.”

189/gene=“FCN3”/site_type=“glycosylation”/note=“N-LINKED (GLCNAC . . . ) (POTENTIAL).”

1 mdllwilpsl willlggpac lktqehpscp gpreleaskv vllpscpgap gspgekgapg

61 pqgppgppgk mgpkgepgdp vnllrcqegp mcrellsqg atlsgwyhlc lpegralpvf

121 cdmdtegggw lvfqrrqdgs vdffrswssy ragfgnqese fwlgnenthq ltlqgnwelr

181 veledfngnr ffahyaffrl lgevdhyqla lgkfsegtag dslslhsgrp fttydadhds

241 snsncavivh gawwyascyr sningryavs daaahkygid wasgrgvghp yrrvrmmlr

XP_{—}130120. similar to Ficolin 2 precursor (Collagen/fibrinogen domain-containing protein 2) (Ficolin-B) (Ficolin B) (Serum lectin P35) (EBP-37) (Hucolin) [Mus mus-culus] [gi:20823464]

59 . . . 95/region-name=“Collagen triple helix repeat (20 copies)”/note=“Collagen”/db_xref=“CDD:pfam01391”

59 . . . 89/region_name=“Collagen triple helix repeat (20 copies)”/note=“Collagen”/db_xref=“CDD:pfam01391”

60 . . . 95/region-name=“Collagen triple helix repeat (20 copies)”/note=“Collagen”/db_xref=“CDD:pfam01391”

60 . . . 95/region-name=“Collagen triple helix repeat (20 copies)”/note=“Collagen”/db_xref=“CDD:pfam01391”

60 . . . 95/region_name=“Collagen triple helix repeat (20 copies)”/note=“Collagen”/db_xref=“CDD:pfam01391”

60 . . . 95/region-name=“Collagen triple helix repeat (20 copies)”/note=“Collagen”/db_xref=“CDD:pfam01391”

60 . . . 95/region-name=“Collagen triple helix repeat (20 copies)”/note=“Collagen”/db_xref=“CDD:pfam01391”

61 . . . 95/region-name=“Collagen triple helix repeat (20 copies)”/note=“Collagen”/db_xref=“CDD:pfam01391”

61 . . . 95/region_name=“Collagen triple helix repeat (20 copies)”/note=“Collagen”/db_xref=“CDD:pfam01391”

61 . . . 95/region-name=“Collagen triple helix repeat (20 copies)”/note=“Collagen”/db_xref=“CDD:pfam01391”

103 . . . 312/region_name=“Fibrinogen beta and gamma chains, C-terminal globular domain”/note=“fibrinogen_C”/db_xref=“CDD:pfam00147”

103 . . . 312/region-name=“Fibrinogen-related domains (FReDs)”/note=“FBG”/db_xref=“CDD:smart00186”

1 malgsaalfv ltltvhaagt cpelkyldle gykqltilqg cpglpgaagp kgeagakgdr

61 gesglpgipg kegptgpkgn qgekgirgek gdsgpsqsca tgprtckell tqghfltgwy

121 tiylpdcrpl tylcdmdtdg ggwtvfqrrl dgsvdffrdw tsykrgfgsq Igefwlgndn

181 ihalttqgts elrvdisdfe gkhdfakyss fqiqgeaeky klilgnflgg gagdsitphn

241 nrifstkdqd ndgstsscam gyhgawwysq chtsnlngly lrgphksyan gvnwkswrgy

301 nysckvsemk vrli

NP_-056654. ficolin 2 isoform d precursor; ficolin (collagen/fibrinogen domain-containing lectin) 2 (hucolin); ficolin (collagen/fibrinogen domain-containing lectin) 2; hucolin [Homo sapiens] [gi:8051590]

39 . . . 95/region_name=“collagen-like domain”

1 meldravgvl gaatlllsfl gmawalqaad tcpevkmvgl egsdkitilr gcpglpgapg

61 dkgeagtngk rgergppgpp gkagppgpng apgepqpclt gd

NP_{—}056653. ficolin 2 isoform c precursor; ficolin (collagen/fibrinogen domain-containing lectin) 2 (hucolin); ficolin (collagen/fibrinogen domain-containing lectin) 2; hucolin [Homo sapiens] [gi:8051588]

39 . . . 95/region_name=“collagen-like domain”

102 . . . 143/region_name=“Fibrinogen beta and gamma chains, C-terminal globular domain”/note=“fibrinogen-C”/db_xref=“CDD:pfam00147”

102 . . . 143/region_name=“Fibrinogen-related domains (FReDs)”/note=“FBG”/db_xref=“CDD:smart00186”

1 meldravgvl gaatlllsfl gmawalqaad tcpevkmvgl egsdkltilr gcpglpgapg

61 dkgeagtngk rgergppgpp gkagppgpng apgepqpcit gprtckdlld rghflsgwht

121 iylpdcrpit vlcdmdtdgg gwtvsvglgr ggqpgspggq aahlvgehtl efsillvgds

181 qr

NP_{—}056652. ficolin 2-isoform b precursor; ficolin (collagen/fibrinogen domain-containing lectin) 2 (hucolin); ficolin (collagen/fibrinogen domain-containing lectin) 2; hucolin [Homo sapiens] [gi:8051586]

siq peptide 1 . . . 25

mat peptide 26 . . . 275

60 . . . 275/region-name=“FBG domain”/note=“fibrinogen beta/gamma homology”

64 . . . 275/region_name=“Fibrinogen-related domains (FReDs)”/note=“FBG”/db_xref=“CDD:smart00186”

64 . . . 274/region-name=“Fibrinogen beta and gamma chains, C-terminal globular domain”/note=“fibrinogen_C”/db_xref=“CDD:pfam00147”

1 meldravgvl gaatillsfl gmawalqaad tcpgergppg ppgkagppgp ngapgepqpc

61 ltgprtckdl ldrghflsgw htiylpdcrp ltylcdmdtd gggwtvfqrr vdgsvdfyrd

121 watykqgfgs rlgefwlgnd nihaltaqgt selrvdlvdf ednyqfakyr sfkvadeaek

181 ynivigafve gsagdsltfh nnqsfstkdq dndintgnca vmfqgawwyk nchvsnlngr

241 ylrgthgsfa nginwksgkg ynysykvsem kvrpa

NP_{—}001994. ficolin 1 precursor, ficolin (collagen/flbrinogen domain-containing) 1 [Homo sapiens] [gi:8051584]

siq peptide 1 . . . 27

mat peptide 28 . . . 326

40 . . . 108/region_name=“collagen-like domain”

50 . . . 105/region_name=“Collagen triple helix repeat (20 copies)”/note=“Collagen”/db_xref=“CDD:pfam01391”

51 . . . 107/region-name=“Collagen triple helix repeat (20 copies)”/note=“Collagen”/db_xref=“CDD:pfam01391”

52 . . . 106/region-name=“Collagen triple helix repeat (20 copies)”/note=“Collagen”/db_xref=“CDD:pfam01391”

115 . . . 326/region_name=“FBG domain”/note=“fibrinogen beta/gamma homology”

115 . . . 326/region_name=“Fibrinogen-related domains (FReDs)”/note=“FBG”/db_xref=“CDD:smart00186”

115 . . . 325/region-name=“Fibrinogen beta and gamma chains, C-terminal globular domain”/note=“fibrinogen_C”/db_xref=“CDD:pfam00147” variation 315/db_xref=“dbSNP:1128428” variation 316/db_xref=“dbSNP:1128429” variation 317/db_xref=“dbSNP:1128430”

1 melsgatmar glavllvlfl hiknlpaqaa dtcpevkwg legsdkltil rgcpglpgap

61 gpkgeagvig ergerglpga pgkagpvgpk gdrgekgmrg ekgdagqsqs catgprnckd

121 lldrgyllsg whfiylpdcr pltvicdmdt dgggwtvfqr rmdgsvdfyr dwaaykqgfg

181 sqlgefwlgn dnihaltaqg sselrvdlvd fegnhqfaky ksfkvadeae kyklylgafv

241 ggsagnsitg hnnnffstkd qdndvsssnc aekfqgawwy adchasning lylmgphesy

301 anginwsaak gykysykvse mkvrpa

NP_{—}004099. ficolin 2 isoform a precursor; ficolin (collagen/fibrinogen domain-containing lectin) 2 (hucolin); ficolin (collagen/fibrinogen domain-containing lectin) 2; hucolin [Homo sapiens] [gi:4758348]

siq peptide 1 . . . 25

mat peptide 26 . . . 313

39 . . . 95/region_name=“collagen-like domain”

98 . . . 313/region_name=“FBG domain”/note=“fibrinogen beta/gamma homology”

102 . . . 313/region_name=“Fibrinogen-related domains (FReDs)”/note=“FBG”/db_xref=“CDD:smart00186”

102 . . . 312/region_name=“Fibrinogen beta and gamma chains, C-terminal globular domain”/note=“fibrinogen_C”/db_xref=“CDD:pfam00147

1 meldravgvl gaatillsfl gmawalqaad tcpevkmvgl egsdkltilr gcpglpgapg

61 dkgeagtngk rgergppgpp gkagppgpng apgepqpcit gprtckdlld rghflsgwht

121 iylpdcrplt vlcdmdtdgg gwtvfqrrvd gsvdfyrdwa tykqgfgsrl gefwlgndni

181 haltaqgtse lrvdlvdfed nyqfakyrsf kvadeaekyn lylgafvegs agdsltfhnn

241 qsfstkdqdn dlntgncavm fqgawwyknc hvsningryl rgthgsfang inwksgkgyn

301 ysykvsemkv rpa

Q9WTS8. Ficolin 1 precursor (Collagen/fibrinogen domain-containing protein 1) (Ficolin-A) (Ficolin A) (M-Ficolin) [gi: 13124116]

1 . . . 22/gene=“FCN1”/region-name=“Signal”/note=“POTENTIAL.”

23 . . . 335/gene=“FCN1”/region_name=“Mature chain”/note=“FICOLIN 1.”

50 . . . 88/gene=“FCN1”/region_name=“Domain”/note=“COLLAGEN-LIKE.”

152 . . . 298/gene=“FCN1”/region_name=“Domain”/note=“FIBRINOGEN C-TERMINAL.”

271/gene=“FCN1”/sitetype=“glycosylation”/note=“N-LINKED (GLCNAC . . . ) (POTENTIAL).”

1 mwwpmlwafp vilcicssqa lgqesgacpd vkivglgaqd kvaviqscps fpgppgpkge

61 pgspagrgeir glqgspgkmg ppgskgepgt mgppgvkgek gergtasplg qkelgdalcr

121 rgprsckdll trgifltgwy tiylpdcrpl tylcdmdvdg ggwtvfqrrv dgsinfyrdw

181 dsykrgfgnl gtefwlgndy lhiltangnq elrvdirefq gqtsfakyss fqvsgeqeky

241 klugqfleg tagdsitkhn nmafsthdqd ndtnggknca alfhgawwyh dchqsnlngr

301 ylpgshesya dginwlsgrg hrysykvaem kiras

Q15485. Ficolin 2 precursor (Collagen/fibrinogen domain-containing protein 2) (Ficolin-B) (Ficolin B) (Serum lectin P35) (EBP-37) (Hucolin) (L-Ficolin) [gi:13124203]

1 . . . 25/gene=“FCN2”/region_name=“Signal”/note=“POTENTIAL.”

26 . . . 313/gene=“FCN2”/region_name=“Mature chain”/note=“FICOLIN 2.”

54 . . . 92/gene=“FCN2”/region_name=“Domain”/note=“COLLAGEN-LIKE.”

131 . . . 277/gene=“FCN2”/region_name=“Domain”/note=“FIBRINOGEN C-TERMINAL.”

240/gene=“FCN2”/site_type=“glycosylation”/note=“N-LINKED (GLCNAC . . . ) (POTENTIAL).”

300/gene=“FCN2”/site_type=“glycosylaton”/note=“N-LINKED (GLCNAC . . . ) (POTENTIAL).”

1 meldravgvl gaatlllsfl gmawalqaad tcpevkmvgl egsdkltilr gcpglpgapg

61 dkgeagtngk rgergppgpp gkagppgpng apgepqpclt gprtckdlld rghflsgwht

121 iylpdcrplt vlcdmdtdgg gwtvfqrrvd gsvdfyrdwa tykqgfgsrl gefwlgndni

181 haltaqgtse lrvdlvdfed nyqfakyrsf kvadeaekyn lvlgafvegs agdsltfhnn

241 qsfstkdqdn dlntgncavm fqgawwyknc hvsningryl rgthgsfang inwksgkgyn

301 ysykvsemkv rpa

O70497. Ficolin 2 precursor (Collagen/fibrinogen domain-containing protein 2) (Ficolin-B) (Ficolin B) (Serum lectin P35) (EBP-37) (Hucolin) [gi:13124181]

<1 . . . 15/gene=“FCN2”/region_name=“Signal”/note=“POTENTIAL.”

16 . . . >306/gene=“FCN2”/region-name=“Mature chain”/note=“FICOLIN 2.”

41 . . . 79/gene=“FCN2”/region_name=“Domain”/note=“COLLAGEN-LIKE.”

130 . . . 276/gene=“FCN2”/region_name=“Domain”/note=“FIBRINOGEN C-TERMINAL.”

299/gene=“FCN2”/sitetype=“glycosylation”/note=“N-LINKED (GLCNAC . . . ) (POTENTIAL).”

1 lgsaalfvlt ltvhaagtcp elkyldlegy kqltilqgcp glpgaagpkg eagakgdrge

61 sglpgipgke gptgpkgnqg ekgirgekgd sgpsqscatg prtckelltq ghfitgwyti

121 ylpdcrpmtv lcdmdtdggg wtvfqrrldg svdffrdwts ykrgfgsqlg efwIgndnih

181 alttqgtsel rvdisdfegk hdfakyssfq iqgeaekykl ilgnflggga gdsltphnnr

241 lfstkdqdnd gstsscamgy hgawwysqch tsninglylr gphksyangv nwkswrgyny

301 sckvse

O70165. Ficolin 1 precursor (Collagen/fibrinogen domain-containing protein 1) (Flcolin-A) (Ficolin A) (M-Ficolin) [gi: 13124179]

1 . . . 22/gene=“FCN1”/region_name=“Signal”/note=“POTENTIAL.”

23 . . . 334/gene=“FCN1”/region_name=“Mature chain”/note=“FICOLIN 1.”

50 . . . 88/gene=“FCN1”/region_name=“Domain”/note=“COLLAGEN-LIKE.”

152 . . . 298/gene=“FCN1”/region_name=“Domain”/note=“FIBRINOGEN C-TERMINAL.”

261/gene=“FCN1”/sitetype=“glycosylation”/note=“N-LINKED (GLCNAC . . . ) (POTENTIAL).”

1 rqwptlwafs gilclcpsqa lgqergacpd vkwglgaqd kvvviqscpg fpgpppkge

61 pgspagrger gfqgspgkmg pagskgepgt mgppgvkgek gdtgaapsig ekelgdtlcq

121. rgprsckdll trgifltgwy tihlpdcrpl tylcdmdvdg ggwtvfqrrv dgsidffrdw

181 dsykrgfgnl gtefwlgndy lhlltangnq elrvdlqdfq gkgsyakyss fqvseeqeky

241 kltlgqfleg tagdsltkhn nmsftthdqd ndansmncaa lfhgawwyhn chqsnlngry

301 lsgshesyad ginwgtgqgh hysykvaemk iras

P57756. Ficolin 2 precursor (Collagen/fibrinogen domain-containing protein 2) (Ficolin-B) (Ficolin B) (Serum lectin P35) (EBP-37) (Hucolin) [gi:13124114]

1 . . . 22/gene=“FCN2”/region_name=“Signal”/note=“POTENTIAL.”

23 . . . 319/gene=“FCN2”/region_name=“Mature chain”/note=“FICOLIN 2.”

48 . . . 86/gene=“FCN2”/region_name=“Domain”/note=“COLLAGEN-LIKE.”

137 . . . 283/gene=“FCN2”/region_name=“Domain”/note=“FIBRINOGEN C-TERMINAL.”

306/gene=“FCN2”/sitetype=“glycosylation”/note=“N-LINKED (GLCNAC . . . ) (POTENTIAL).”

1 mvlgsaalfv lslcvteltl haadtcpevk vldlegsnkl tilqgcpglp galgpkgeag

61 akgdrgesgl pghpgkagpt gpkgdrgekg vrgekgdtgp sqscatgprt ckelltrgyf

121 ltgwytiylp dcrpltylcd mdtdgggwtv fqrridgtvd ffrdwtsykq gfgsqlgefw

181 lgndnihalt tqgtnelrvd ladfdgnhdf akyssfqiqg eaekyklilg nflgggagds

241 ltsqnnmlfs tkdqdndqgs sncavryhga wwysdchtsn lnglylrglh ksyangvnwk

301 swkgynysyk vsemkvrli

JC5980: ficolin-A precurs—mouse [gi:7513652]

1 . . . 21/region_name=“domain”/note=“signal sequence”

50 . . . 64/region-name=“domain”/note=“collagen-like”

68 . . . 106/region-name=“domain”/note=“collagen-like”

123 . . . 334/region-name=“domain”/note=“fibrinogen beta/gamma homology #label FBG”

1 mqwptlwafs gllcicpsqa lgqergacpd vkwglgaqd kvvviqscpg fpgppgpkge

61 pgspagrger gfqgspgkmg pagskgepgt mgppgvkgek gdtgaapslg ekelgdtlcq

121 rgprsckdll trgifltgwy tihlpdcrpl tylcdmdvdg ggwtvfqrrv dgsidffrdw

181 dsykrgfgnl gtefwlgndy lhlltangnq elrvdlqdfq gkgsyakyss fqvseeqeky

241 kltlgqfleg tagdsitkhn nmsftthdqd ndansmncaa lfhgawwyhn chqsningry

301 lsgshesyad ginwgtgqgh hysykvaemk iras

S61517. ficolin-1 precurs-human [gi:2135116]

1 . . . 326/note=“36K HLA-cross-reactive plasma protein; hucolin, 35K”

1 . . . 22/region-name=“domain”/note=“signal sequence”

52 . . . 108/region_name=“region”/note=“collagen-like”

115 . . . 326/region-name=“domain”/note=“fibrinogen beta/gamma homology #label FBG”

305/sitetype=“binding”/note=“carbohydrate (Asn) (covalent)”

1 melsgatmar glavilylfl hiknlpaqaa dtcpevkwg legsdkltil rgcpglpgap

61 gpkgeagvig ergerglpga pgkagpvgpk gdrgekgmrg ekgdagqsqs catgprnckd

121 lldrgyflsg whniylpdcr pltylcdmdt dgggwtvfqr rmdgsvdfyr dwaaykqgfg

181 sqlgefwlgn dnihaltaqg sselrvdlvd fegnhqfaky ksfkvadeae kyklylgafv

241 ggsagnsitg hnnnffstkd qdndvsssnc aekfqgawwy adchasslng lylmgphesy

301 anginwsaak gykysykvse mkvrpa

A47172. transforming growth factor-beta 1-binding protein homolog ficolin-alpha pig [gi:423206]

112 . . . 323/region_name=“domain”/note=“fibrinogen beta/gamma homology #label FBG”

1 mdtrgvaaam rplyllvafl ctaapaldtc pevkwgleg sdklsilrgc pglpgaagpk

61 geagasgpkg gqgppgapge pgppgpkgdr gekgepgpkg esweteqclt gprtckellt

121 rghilsgwht iylpdcqplt vlcdmdtdgg gwtvfqrrsd gsvdfyrdwa aykrgfgsql

181 gefwlgndhi haltaqgtne lrvdivdfeg nhqfakyrsf qvadeaekym lvigafvegn

241 agdsltshnn slfttkdqdn dqyasncavl yqgawwynsc hvsningryl ggshgsfang

301 vnwssgkgyn ysykvsemkf rat

JC4942. ficolin-1 precursor—human [gi:2135117]

1 . . . 22/region_name=“domain”/note=“signal sequence”

45 . . . 101/region-name=“region”/note=“collagen-like”

108 . . . 319/region_name=“domain”/note=“fibrinogen beta/gamma homology #label FBG”

111 . . . 315/region_name=“region”/note=“fibrinogen-like”

298/site_type=“binding”/note=“carbohydrate (Asn) (covalent)”

1 marglavllv lflhiknipa qaadtcpevk wglegsdkl tilrgcpglp gapgpkgeag

61 vigergergl pgapgkagpv gpkgdrgekg mrgekgdagq sqscatgprn ckdlldrgyf

121 lsgwhtiylp dcrpltylcd mdtdgggwtv fqrrmdgsvd fyrdwaaykq gfgsqlgefw

181 lgndnihalt aqgsselrvd lvdfegnhqf akyksfkvad eaekyklvig afvggsagns

241 ltghnnnffs tkdqdndvss sncaekfqga wwyadchasn lnglylmgph esyanginws

301 aakgykysyk vsemkvrpa

AAF44911. symbol=BG:DS00929 . . . [gi:7287873]

1 mkscffvlfl wtllfevgqs sphtcpsgsp ngihqlmlpe eepfqvtqck ttardwiviq

61 rrldgsvnfn qswfsykdgf gdpngeffig lqklylmtre qphelfiqlk hgpgatvyah

121 fddfqvdset elyklervgk ysgtagdsir yhinkrfsff drdndesskn caaehgggww

181 fhsclsr

The first polypeptide preferably comprises at least 10, such as at least 12, for example at least 15, such as at least 20, for example at least 25, such as at least 30, for example at least 35, such as at least 40, for example at least 50 consecutive amino acid residues of the complement activating protein or of a variant or a homologue to said protein. Such a variant or homologue is preferably at least 70%, such as 80%, for example 90%, such as 95% identical to the complement activating protein.

The first polypeptide sequence of the fusion protein is preferably capable of activating the lectin-complement pathway when bound directly or indirectly to a target, such as a bacteria or a virus. In a preferred embodiment the first polypeptide sequence is capable of associating with at least one MASP protein, such as a MASP protein selected from the group consisting of MASP-1, MASP-2 and MASP-3 or functional homologues or variants hereof. In particular the first polypeptide is capable of associating with said at least one MASP protein when being part of the fusion protein. Thereby the first polypeptide sequence is capable of providing the fusion protein with complement system activating activity.

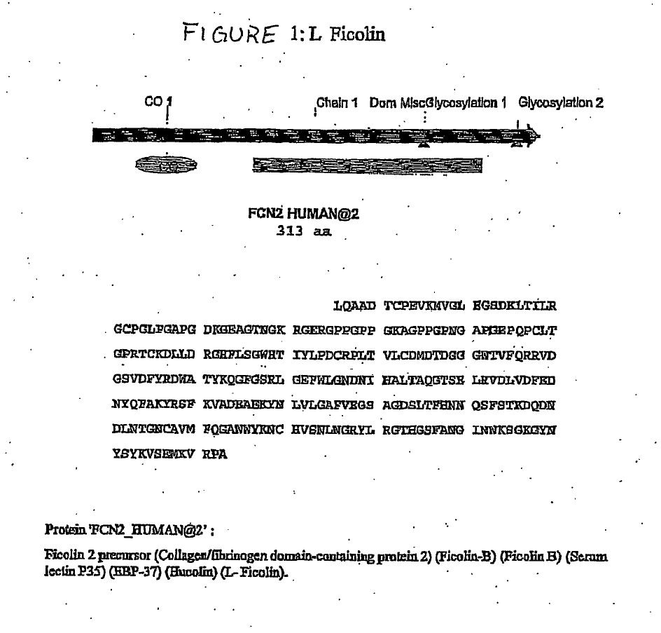

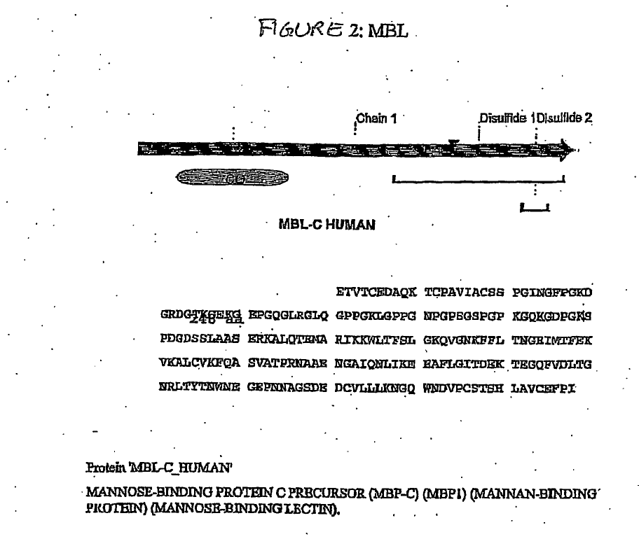

In a particular preferred embodiment the first polypeptide sequence comprises at least the amino acid residues corresponding to 1-54 of L-ficolin sequence of FIG. 1, such as 1-55 of L-ficolin sequence of FIG. 1, such as 1-69 of L-ficolin sequence of FIG. 1, such as 1-77 of L-ficolin sequence of FIG. 1, such as 1-90 of L-ficolin sequence of FIG. 1, such as 1-93 of L-ficolin sequence of FIG. 1, such as 1-131 of L-ficolin sequence of FIG. 1, such as 1-207 of L-ficolin sequence of FIG. 1. In particular the first polypeptide sequence comprises the amino acid residues selected from: 1-55 of L-ficolin sequence of FIG. 1, 1-54 of L-ficolin sequence of FIG. 1, 1-50, or 1-77 of L-ficolin sequence of FIG. 1. In a more preferred embodiment the first polypeptide sequence has the amino acid residues selected from: 1-55 of L-ficolin sequence of FIG. 1, 1-54 of L-ficolin sequence of FIG. 1, 1-50, or 1-77 of L-ficolin sequence of FIG. 1. In another embodiment the first polypeptide sequence comprises at least the amino acid residues corresponding to 60-90 of L-ficolin sequence of FIG. 1, such as 55-90 of L-ficolin sequence of FIG. 1, such as 54-92 of L-ficolin sequence of FIG. 1.

It is preferred the first polypeptide sequence and the second polypeptide sequence are selected to include the motif X-X-G-X-X-G at least 5 times, such as at least 7 times, preferably in a consecutive sequence. It is more preferred to select the first polypeptide sequence and the second polypeptide sequence so that the aforementioned motif is substituted once with the motif X-X-G-X-G. In the motifs X means any amino acid different from Glycine, and G means Glycine.

Second Polypeptide Sequence

The second polypeptide sequence is preferably capable of associating with one or more carbohydrates. This may be accomplished by incorporating at least the carbohydrate recognizing domain of the collectin in question. Accordingly, the second polypeptide sequence preferably comprises the CRD domain of a collectin or a homologue or a variant thereof.

Preferably the collectin is selected from the group consisting of MBL (mannose-binding lectin), SP-A (lung surfactant protein A), SP-D (lung surfactant protein D), BK (or BC, bovine conglutinin) and CL-43 (collectin-43). Most preferably the collectin is MBL.

In a particular preferred embodiment the collectin has one of the sequences listed below with reference to their database and accession No.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

The second polypeptide preferably comprises at least 10, such as at least 12, for example at least 15, such as at least 20, for example at least 25, such as at least 30, for example at least 35, such as at least 40, for example at least 50 consecutive amino acid residues of the collectin or of a variant or a homologue to said protein. Such a variant or homologue is preferably at least 70%, such as 80%, for example 90%, such as 95% identical to the collectin.

In a preferred embodiment the second polypeptide sequence comprises the CRD domain of MBL or the neck region of MBL or the collagen-like domain of MBL. More preferably the second polypeptide comprises the neck region and the CRD domain of MBL. In a most preferred embodiment the second polypeptide sequence comprises the collagen-like domain, the neck region and the CRD domain of MBL. MBL is as defined above.

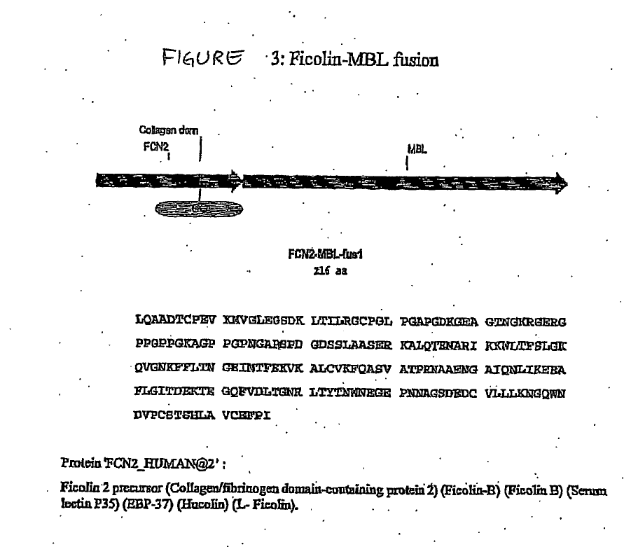

Preferably the second polypeptide sequence comprises at least amino acids 170-200 of the MBL sequence shown in FIG. 2, such as at least amino acids 160-200 of the MBL sequence shown in FIG. 2, such as at least amino acids 150-200 of the MBL sequence shown in FIG. 2, such as at least amino acids 140-200 of the MBL sequence shown in FIG. 2, such as at least amino acids 130-200 of the MBL sequence shown in FIG. 2, such as at least amino acids 120-200 of the MBL sequence shown in FIG. 2, such as at least amino acids 110-200 of the MBL sequence shown in FIG. 2, such as at least amino acids 100-200 of the MBL sequence shown in FIG. 2, such as at least amino acids 90-200 of the MBL sequence shown in FIG. 2, such as at least amino acids 80-200 of the MBL sequence shown in FIG. 2, such as at least amino acids 70-200 of the MBL sequence shown in FIG. 2, such as at least amino acids 60-200 of the MBL sequence shown in FIG. 2, such as at least amino acids 80-228 of the MBL sequence shown in FIG. 2.

Preferably the second polypeptide sequence comprises amino acids 80-228 of SEQ ID. NO 2.

In a preferred embodiment the second polypeptide sequence is capable of associating with at least one MASP protein, such as a MASP protein selected from the group consisting of MASP-1, MASP-2 and MASP-3 or functional homologues or variants hereof. In particular the second polypeptide is capable of associating with said at least one MASP protein when being part of the fusion protein. Thereby the second polypeptide sequence is capable of providing the fusion protein with complement system activating activity. In a preferred embodiment the second polypeptide sequence comprises an amino acid sequence selected from: 56-228 of SEQ ID. NO 2, 55-228 of SEQ ID. NO 2, 54-228 of SEQ ID. NO 2, and 50-228 of SEQ ID. NO 2. In a preferred embodiment the second polypeptide sequence has an amino acid sequence selected from: 56-228 of SEQ ID. NO 2, 55-228 of SEQ ID. NO 2, 54-228 of SEQ ID. NO 2, and 50-228 of SEQ ID. NO-2.

In another embodiment the second polypeptide comprises the cysteine-rich region of the collectin, such as the N-terminal region of the collectin.

Fusion Protein

The fusion protein comprises the first and the second polypeptide connected to each other, optionally through a linker region. In a preferred embodiment the first polypeptide sequence is positioned N-terminally in the fusion protein and the second polypeptide sequence is positioned C-terminally.

Specific examples of the components of the fusion protein are:

A fusion protein comprising the cysteine-rich region and the collagen-like domain of L-ficolin and the CRD domain of MBL.

A fusion protein comprising the cysteine-rich region of L-ficolin and the collagen-like domain, the neck region and the CRD domain of MBL.

A fusion protein comprising the cysteine-rich region and the collagen-like domain of H-ficolin and the CRD domain of MBL.

A fusion protein comprising the cysteine-rich region of H-ficolin and the collagen-like domain, the neck region and the CRD domain of MBL.

A fusion protein-comprising the cysteine-rich region and the collagen-like domain of M-ficolin and the CRD domain of MBL.

A fusion protein comprising the cysteine-rich region of M-ficolin and the collagen-like domain, the neck region and the CRD domain of MBL.

A fusion protein comprising the cysteine-rich region of MBL, and the CRD domain of ficolin.

A fusion protein comprising the cysteine-rich region of MBL and the collagen-like domain, the neck region and the CRD domain of ficolin.

A fusion protein comprising the cysteine-rich region and the collagen-like domain of L-ficolin and the CRD domain of Pulmonary surfactant-associated protein D.

A fusion protein comprising the cysteine-rich region of L-ficolin and the collagen-like domain, the neck region and the CRD domain of Pulmonary surfactant-associated protein D.

A fusion protein comprising the cysteine-rich region and the collagen-like domain of a ficolin and the CRD domain of a collectin-43.

A fusion protein comprising the cysteine-rich region of a ficolin and the collagen-like domain, the neck region and the CRD domain of a collectin-43.

A fusion protein comprising the amino acid sequence as defined by the sequence shown in FIG. 3, or a functional homologue thereof, preferably a fusion protein consisting of the amino acid sequence as shown in FIG. 3. In another embodiment the fusion protein has amino acid sequence 1-50 of the amino acid shown in FIG. 1 and amino acid sequence 54-228 of the amino acid sequence shown in FIG. 2.

As discussed above the fusion protein is preferably capable of forming subunit complexes as well as oligomers of subunit complexes. Preferably the fusion protein forms substantially only trimeric, tetrameric, pentameric and hexameric subunit oligomers, such as trimeric, tetrameric, and pentameric subunit oligomers, such as trimeric or tetrameric subunit oligomers, more preferably substantially only tetrameric subunit oligomers, in order to obtain a more homogenous composition of fusion proteins.

Homologues

In the present context the terms homologue or variant or functional homologues are used as synonymes, wherein a homologue of a protein exhibits one or more substitutions, deletions, and/or additions of one or more amino acid residues. Fragments are a subgroup of homologues being truncations of the protein.

A homologue of the protein may comprise one or more conservative amino acid substitutions, such as at least 2 conservative amino acid substitutions, for example at least 3 conservative amino acid substitutions, such as at least 5 conservative amino acid substitutions, for example at least 10 conservative amino acid substitutions, such as at least 20 conservative amino acid substitutions, for example at least 50 conservative amino acid substitutions such as at least 75 conservative amino acid substitutions, for example at least 100 conservative amino acid substitutions. Conservative amino acid substitutions within the meaning of the present invention is substitution of one amino acid within a predetermined group of amino acids for another amino acid within the same predetermined group, exhibiting similar or substantially similar characteristics. Such predetermined groups are for example:

polar side chains (Asp, Glu, Lys, Arg, His, Asn, Gln, Ser, Thr, Tyr, and Cys)

non-polar side chains (Gly, Ala, Val, Leu, Ile, Phe, Trp, Pro, and Met)

aliphatic side chains (Gly, Ala Val, Leu, Ile)

cyclic side chains (Phe, Tyr, Trp, His, Pro)

aromatic side chains (Phe, Tyr, Trp)

acidic side chains (Asp, Glu)

basic side chains (Lys, Arg, His)

amide side chains (Asn, Gin)

hydroxy side chains (Ser, Thr)

sulphor-containing side chains (Cys, Met), and

amino acids being monoamino-dicarboxylic acids or monoamino-monocarboxylic-monoamidocarboxylic acids (Asp, Glu, Asn, Gin).

Conservative substitutions may be introduced in any position of a preferred protein. It may however also be desirable to introduce non-conservative substitutions. A non-conservative substitution should lead to the formation of a homologue of a protein capable of exerting a function similar to the function of said protein. Such substitution could for example i) differ substantially in hydrophobicity, for example a hydrophobic residue (Val, Ile, Leu, Phe or Met) substituted for a hydrophilic residue such as Arg, Lys, Trp or Asn, or a hydrophilic residue such as Thr, Ser, His, Gin, Asn, Lys, Asp, Glu or Trp substituted for a hydrophobic residue; and/or ii) differ substantially in its effect-on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

In a further embodiment the present invention relates to homologues of a preferred protein, wherein such homologues comprise substituted amino acids having hydrophilic or hydropathic indices that are within +/−2.5, for example within +/−2.3, such as within +/−2.1, for example within +/−2.0, such as within +/−1.8, for example within +/−1.6, such as within +/−1.5, for example within +/−1.4, such as within +/−1.3 for example within +/−1.2, such as within +/−1.1, for example within +/−1.0, such as within +/−0.9, for example within +/−0.8, such as within +/−0.7, for example within +/−0.6, such as within +/−0.5, for example within +/−0.4, such as within +/−0.3, for example within +/−0.25, such as within +/−0.2 of the value of the amino acid it has substituted.

The importance of the hydrophilic and hydropathic amino acid indices in conferring interactive biologic function on a protein is well understood in the art (Kyte & Doolittle, 1982 and Hopp, U.S. Pat. No. 4,554,101, each incorporated herein by reference).

The amino acid hydropathic index values as used herein are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5) (Kyte & Doolittle, 1982).

The amino acid hydrophilicity values are: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4) (U.S. Pat. No. 4,554,101).

Substitution of amino acids can therefore in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

Furthermore, a homologue may comprise addition or deletion of an amino acid, for example an addition or deletion of from 2 to 100 amino acids, such as from 2 to 50 amino acids, for example from 2 to 20 amino acids, such as from 2 to 10 amino acids, for example from 2 to 5 amino acids, such as from 2 to 3 amino acids. However, additions of more than 100 amino acids, such as additions from 100 to 500 amino acids, are also comprised within the present invention.

Proteins sharing at least some homology with a preferred protein are to be considered as falling within the scope of the present invention when they are at least about 40 percent homologous, or preferably identical, with the preferred protein, such as at least about 50 percent homologous, or preferably identical, for example at least about 60 percent homologous, or preferably identical, such as at least about 70 percent homologous, or preferably identical, for example at least about 75 percent homologous, or preferably identical, such as at least about 80 percent homologous, or preferably identical, for example at least about 85 percent homologous, or preferably identical, such as at least about 90 percent homologous, or preferably identical, for example at least 92 percent homologous, or preferably identical, such as at least 94 percent homologous, or preferably identical, for example at least 95 percent homologous, or preferably identical, such as at least 96 percent homologous, or preferably identical, for example at least 97 percent homologous, s or preferably identical, uch as at least 98 percent homologous, or preferably identical, for example at least 99 percent homologous, or preferably identical, with the preferred protein.

Preferred proteins are complement activating proteins comprising collecting and lectins and homologues hereof.

Homologues of Collecting

A homologue of a collectin including MBL within the scope of the present invention should be understood as any protein capable of exerting a function similar to the function of a collectin and comprising one or more of the variations described above. In particular such function is the ability to activate complement upon binding to one or more carbohydrates.

The terms functional homologues of collectin used herein relate to functional equivalents or a fragment of collectin comprising a predetermined amino acid sequence, and such homologues are defined as:
a) A homologue comprising an amino acid sequence capable of recognising and binding to glucans, lipophosphoglycans and glycoinositol phospholipids that contain sugar with 3- and 4-hydroxyl groups in the pyranose ring (i.e. Man, Glc, Fuc or GlcNAc) either alone or when being subunit complexed as described above and/or
b) A homologue comprising an amino acid sequence capable of forming an association with a component of the Lectin/MBL pathway such as binding to the MASP-1, MASP-2, MASP-3 and/or sMAP either alone or when being subunit complexed as described above, wherein said binding result in activation of the Lectin/MBL pathway and/or
c) A homologue comprising an amino acid sequence capable of by the collagen-like domain forming an oligomeric structure of two or more subunits, where a subunit comprises three identical polypeptides of a cysteine-rich region, a collagen-like domain, a neck region and a carbohydrate recognition domain.
   Homologues of Lectins

A homologue of a lectin including ficolins within the scope of the present invention should be understood as any protein capable of exerting a function similar to the function of a lectin and comprising one or more of the variations previously described. In particular such function is the ability to activate complement upon binding to one or more carbohydrates.

The terms functional homologues of, lectin used herein relate to functional equivalents of a fragment of lectin comprising a predetermined amino acid sequence, and such homologues are defined as:
a) A homologue comprising an amino acid sequence capable of recognising and binding to N-acetyl-glucosamine (GlcNAc), or N-acetyl-galactosamine (GalNAc), or elastin either alone or when being subunit complexed as described above and/or
b) A homologue comprising an amino acid sequence capable of forming an association with a component of the Lectin/MBL pathway such as binding to the MASP-1, MASP-2, MASP-3 and/or sMAP either alone or when being subunit complexed as described above, wherein said binding result in activation of the Lectin/MBL pathway and/or
c) A homologue comprising an amino acid sequence capable of by the collagen-like domain forming an oligomeric structure of two or more subunits, where a subunit comprises three identical polypeptides of a cysteine-rich region, a collagen-like domain, a neck region and a fibrinogen-like domain.

The activation of the lectin/MBL pathway, i.e. the activity of the fusion protein to activate the complement system may be assessed by assessing the C4 cleaving effect of the fusion protein or subunit complexes or oligomers of complexes thereof by the following method comprising the steps of
applying a sample comprising a predetermined amount of fusion protein as well as a predetermined amount of MASP-1, MASP-2 or MASP-3,
applying at least one complement factor to the sample,
detecting the amount of cleaved complement factors,
correlating the amount of cleaved complement factors to the amount of fusion protein, and
determining the activity of the fusion protein.

The complement factor preferably used in the present method is a complement factor cleavable by the MBL/MASP-2 complex, such as C4. However, the complement factor may also be selected from C3 and C5.

The cleaved complement factor may be detected by a variety of means, such as by of antibodies directed to the cleaved complement factor.

The assay is carried out at conditions which minimize or eliminate interference from the classical complement activation pathway and the alternative complement activation pathway.

Preferably a homologue of a collectin and/or a lectin exhibits two of the functions defined above; more preferably three of the functions defined above.

Preparation of Fusion Protein

The fusion protein may be prepared by any suitable method known to the person skilled in the art. Below are described several of the methods for preparing the fusion protein, however the invention is not limited to those, methods.

Synthetic Preparation

When appropriate, in particular in relation to the size of the fusion protein, the fusion protein may be produced synthetically. The methods for synthetic production of peptides are well known in art. Detailed descriptions as well as practical advice for producing synthetic peptides may be found in Synthetic Peptides: A User's Guide (Advances in Molecular Biology), Grant G. A ed., Oxford University Press, 2002, or in: Pharmaceutical Formulation: Development of Peptides and Proteins, Frokjaer and Hovgaard eds., Taylor and Francis, 1999.

Recombinant Preparation

The fusion proteins of the invention are preferably produced by use of recombinant DNA technologies. The DNA sequence encoding each part of the fusion protein may be prepared by fragmentation of the DNA sequences encoding the full-length protein, (genomic DNA or cDNA) which the fusion protein part is derived from, using DNAase I according to a standard protocol (Sambrook et al., Molecular cloning: A Laboratory manual. 2 rd ed., CSHL Press, Cold Spring Harbor, N.Y., 1989). The obtained DNA sequences encoding the individual parts of the fusion protein may then be fused together.

The DNA sequence may also be prepared by polymerase chain reaction using specific primers, for instance as described in U.S. Pat. No. 4,683,202 or Saiki et al., 1988, Science 239:487-491.

The DNA sequence encoding a fusion protein of the invention may be prepared synthetically by established standard methods, e.g. the phosphoamidine method described by Beaucage and Caruthers, 1981, Tetrahedron Lett. 22:1859-1869, or the method described by Matthes et al., 1984, EMBO J. 3:801-805. According to the phosphoamidine method, oligonucleotides are synthesized, e.g. in an automatic DNA synthesizer, purified, annealed, ligated and cloned in suitable vectors.

The DNA sequence is then inserted into a recombinant expression vector, which may be any vector, which may conveniently be subjected to recombinant DNA procedures. The choice of vector will often depend on the host cell into which it is to be introduced. Thus, the vector may be an autonomously replicating vector, i.e. a vector that exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

In the vector, the DNA sequence encoding a fusion protein should be operably connected to a suitable promoter sequence. The promoter may be any DNA sequence, which shows transcriptional activity in the host cell of choice and may be derived from genes encoding proteins either homologous or heterologous to the host cell. Examples of suitable promoters for directing the transcription of the coding DNA sequence in mammalian cells are the SV 40 promoter (Subramani et al., 1981, Mol. Cell Biol. 1:854-864), the MT-1 (metallothionein gene) promoter (Palmiter et al., 1983, Science 222: 809-814) or the adenovirus 2 major late promoter. A suitable promoter for use in insect cells is the polyhedrin promoter (Vasuvedan et al., 1992, FEBS Lett. 311:7-11). Suitable promoters for use in yeast host cells include promoters from yeast glycolytic genes (Hitzeman et al., 1980, J. Biol. Chem. 255:12073-12080; Alber and Kawasaki, 1982, J. Mol. Appl. Gen. 1: 419-434) or alcohol dehydrogenase genes (Young et al., 1982, in Genetic Engineering of Microorganisms for Chemicals, Hollaender et al, eds., Plenum Press, New York), or the TPI1 (U.S. Pat. No. 4,599,311) or ADH2-4c (Russell et al., 1983, Nature 304:652-654) promoters. Suitable promoters for use in filamentous fungus host cells are, for instance, the ADH3 promoter (McKnight et al, 1985, EMBO J. 4:2093-2099) or the tplA promoter.

The coding DNA sequence may also be operably connected to a suitable terminator, such as the human growth hormone terminator (Palmiter et al., op. cit.) or (for fungal hosts) the TPI1 (Alber and Kawasaki, op. cit.) or ADH3 (McKnight et al., op. cit.) promoters. The vector may further comprise elements such as polyadenylation signals (e.g. from SV 40 or the adenovirus 5 Elb region), transcriptional enhancer sequences (e.g. the SV 40 enhancer) and translational enhancer sequences (e.g. the ones encoding adenovirus VA RNAs).

The recombinant expression vector may further comprise a DNA sequence enabling the vector to replicate in the host cell in question. An example of such a sequence (when the host cell is a mammalian cell) is the SV 40 origin of replication. The vector may also comprise a selectable marker, e.g. a gene the product of which complements a defect in the host cell, such as the gene coding for dihydrofolate reductase (DHFR) or one which confers resistance to a drug, e.g. neomycin, hydromycin or methotrexate.

The procedures used to ligate the DNA sequences coding the fusion proteins, the promoter and the terminator, respectively, and to insert them into suitable vectors containing the information necessary for replication, are well known to persons skilled in the art (cf., for instance, Sambrook et al., op. cit).

The synthesis of the recombinant fusion protein may be by use of in vitro or in vivo cultures. The host cell culture is preferably an eucaryotic host cell culture. By transformation of an eukaryotic cell culture is in this context meant introduction of recombinant DNA into the cells. The expression construct used in the process is characterised by having the encoding region selected from mammalian genes including human genes and genes with big resemblance herewith such as the genes from the chimpanzee. The expression construct used is furthermore featured by the promoter region being selected from genes of virus or eukaryotes, including mammalian cells and cells from insects.

The process for producing recombinant MBL according to the invention is characterised in that the host cell culture is preferably eukaryotic, and for example a mammalian cell culture. A preferred host cell culture is a culture of human kidney cells and in an even more preferred form the host cell culture is a culture of human embryonal kidney cells (HEK cells), such as HEK 293 cell lines for production of recombinant human MBL. By “HEK 293 cell lines” is meant any cell line derived from human embryonal kidney tissue such as, but not limited to, the cell lines deposited at the American Type Culture Collection with the numbers CRL-1573 and CRL-10852.

Other cells may be chick embryo fibroblast, hamster ovary cells, baby hamster kidney cells, human cervical carcinoma cells, human melanoma cells, human kidney cells, human umbilical vascular endothelium cells, human brain endothelium cells, human oral cavity tumor cells, monkey kidney cells, mouse fibroblast, mouse kidney cells, mouse connective tissue cells, mouse oligodendritic cells, mouse macrophage, mouse fibroblast, mouse neuroblastoma cells, mouse pre-B cell, mouse B lymphoma cells, mouse plasmacytoma cells, mouse teratocacinoma cells, rat astrocytoma cells, rat mammary epithelium cells, COS, CHO, BHK, VERO, HeLa, MDCK, WI38, and NIH 3T3 cells.

Alternatively, fungal cells (including yeast cells) may be used as host cells. Examples of suitable yeast cells include cells of Saccharomyces spp. or Schizosaccharomyces spp. in particular strains of Saccharomyces cerevisiae. Examples of other fungal cells are cells of filamentous fungi, e.g. Aspergillus spp. or Neurospora spp., in particular strains of Aspergillus oryzae or Aspergillus niger. The use of Aspergillus spp. for the expression of proteins is described in, e.g., EP 238 023.

In addition, a host cell strain may be chosen which modulates the expression of the inserted sequences, or modifies and processes the gene product in the specific fashion desired. Such modifications (for example, glycosylation) and processing (for example, cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. The mammalian cell types listed above are among those that could serve as suitable host cells.

Methods of transfecting mammalian cells and expressing DNA sequences introduced in the cells are described in e.g. Kaufman and Sharp, J. Mol. Biol. 159, 1982, pp. 601-621; Southern and Berg, 1982, J. Mol. Appl. Genet. 1:327-341; Loyter et al.,. 1982, Proc. Natl. Acad. Sci. USA 79: 422-426; Wigler et al., 1978, Cell 14:725; Corsaro and Pearson, 1981, in Somatic Cell Genetics 7, p. 603; Graham and van der Eb, 1973, Virol. 52:456; and Neumann et al., 1982, EMBO J. 1:841-845.

Other eucaryotic production systems are also envisaged by the present invention, such as the production of the fusion protein in a transgenic plant or animal.

In another aspect the present invention provides a method for producing a fusion protein by

preparing a gene expression construct as defined above encoding a fusion protein,

transforming a host cell culture with the construct,

cultivating the host cell culture, thereby obtaining expression and secretion of the polypeptide into the culture medium, followed by

obtaining a culture medium comprising recombinant fusion protein, and

purifying the fusion protein.

The medium used to culture the cells may be any conventional medium suitable for growing mammalian cells, such as a serum-containing or serum-free medium containing appropriate supplements, or a suitable medium for growing insect, yeast or fungal cells. Suitable media are available from commercial suppliers or may be prepared according to published recipes (e.g. in catalogues of the American Type Culture Collection). Example of culture medium are RPMI-1640 or DMEM supplemented with, e.g., insulin, transferrin, selenium, and foetal bovine serum

The fusion proteins recombinantly produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. HPLC, ion exchange chromatography, affinity chromatography, or the like.

In a preferred embodiment the fusion protein is purified by use of carbohydrate affinity chromatography as described above. In a preferred embodiment of the invention the affinity chromatography is performed by means of matrices of mannose, hexose or N-acetyl-glucoseamin derivatized matrices, which are suitable for affinity chromatography. In particular, an affinity chromatography is used, in which the matrices have been derivatizised with mannose.

Purified recombinant fusion protein is in this context to be understood as recombinant fusion protein purified from cell culture supernatants or body fluids or tissue from transgenic animals purified by use of for example carbohydrate affinity chomatography.

After application of the culture media the column is washed, preferably by using non-denaturing buffers, having a composition, pH and ionic strength resulting in elimination of proteins, without eluting the fusion protein. Such a buffer may be TBS. Elution of fusion protein is performed with a selective desorbing agent, capable of efficient elution of fusion protein, such as TBS containing a desorbing agent, such as EDTA (5 mM for example) or mannose (50 mM for example), and fusion proteins are collected.

Pharmaceutical Composition and Treatment

The fusion protein obtained by the present invention may be used for the preparation of a pharmaceutical composition for the prevention and/or treatment of various diseases or conditions. In the present context the term pharmaceutical composition is used synonymously with the wording medicament.

In addition to the fusion protein, the pharmaceutical composition may comprise a pharmaceutically acceptable carrier substance and/or vehicles.

In particular, a stabilising agent may be added to stabilise the fusion proteins. The stabilising agent may be a sugar alcohol, saccharide, protein and/or amino-acids. An example of a stabilising agent may be albumin or maltose.

Other conventional additives may be added to the pharmaceutical composition depending on administration form for example.

In one embodiment the pharmaceutical composition is in a form suitable for injections. Conventional carrier substances, such as isotonic saline, may be used.

In another embodiment the pharmaceutical composition is in a form suitable for pulmonal administration, such as in the form of a powder for inhalation or creme or fluid for topical application.

A treatment in this context may comprise cure and/or prophylaxis of e.g. the immune system and reproductive system by humans and by animals having said functional units acting in this respect like those in humans. By conditions to be treated are not necessarily meant conditions presently known to be in a need of treatment, but comprise generally any condition in connection with current and/or expected need or in connection with an improvement of a normal condition. In particular, the treatment is a treatment of a condition of deficiency of lectins, such as MBL deficiency.

In another aspect of the present invention the manufacture is provided of a medicament consisting of said pharmaceutical compositions of fusion protein intended for treatment of conditions comprising cure and/or prophylaxis of conditions of diseases and disorders of e.g. the immune system and reproductive system by humans and by animals having said functional units acting like those in humans.

Said diseases, disorders and/or conditions in need of treatment with the compounds of the invention comprise eg treatment of conditions of deficiency of MBL, treatment of cancer and of infections in connection with immunosuppressive chemotherapy including in particular those infections which are seen in connection with conditions during cancer treatment or in connection with implantation and/or transplantation of organs. The invention also comprises treatment of conditions in connection with recurrent miscarriage.

Thus, in particular the pharmaceutical composition may be used for the treatment and/or prevention of clinical conditions selected from infections, MBL deficiency, cancer, disorders associated with chemotherapy, such as infections, diseases associated with human immunodeficiency virus (HIV), diseases related with congenital or acquired immunodeficiency. More particularly, chronic inflammatory demyelinating polyneuropathy (CIDP, Multifocal motoric neuropathy, Multiple scelrosis, Myasthenia Gravis, Eaton-Lamberts syndrome, Opticus Neuritis, Epilepsy; Primary antiphosholipid syndrome; Rheumatoid arthritis, Systemic Lupus erythematosus, Systemic scleroderma, Vasculitis, Wegner's granulomatosis, Sjøgren's syndrome, Juvenile rheumatiod arthritis; Autoimmune neutropenia, Autoimmune haemolytic anaemia, Neutropenia; Crohn's disease, Colitis ulcerous, Coeliac disease; Asthma, Septic shock syndrome, Chronic fatigue syndrome, Psoriasis, Toxic shock syndrome, Diabetes, Sinuitis, Dilated cardiomyopathy, Endocarditis, Atherosclerosis, Primary hypo/agammaglobulinaemia including common variable immunodeficiency, Wiskot-Aldrich syndrome and serve combined immunodefiency (SCID), Secondary hypo/agammaglobulinaemia in patients with chronic lymphatic leukaemia (CLL) and multiple myeloma, Acute and chronic idiopathic thrombocytopenic purpura-(ITP), Allogenic bone marrow transplantation (BTM), Kawasaki's disease, and Guillan-Barre's syndrome.

The route of administration may be any suitable route, such as intravenously, intramusculary, subcutanously or intradermally. Also, pulmonal or topical administration is envisaged by the present invention.

In particular the fusion protein may be administered to prevent and/or treat infections in patients having clinical symptoms associated with congenital or acquired MBL deficiency or being at risk of developing such symptoms. A wide variety of conditions may lead to increased susceptibility to infections in MBL-deficient individuals, such as chemotherapy or other therapeutic cell toxic treatments, cancer, AIDS, genetic disposition, chronic infections, and neutropenia.

The pharmaceutical composition may thus be administered for a period before the onset of administration of chemotherapy or the like and during at least a part of the chemotherapy.

The fusion protein may be administered as a general “booster” before chemotherapy, or it may be administered to those only being at risk of developing MBL deficiency. The group of patients being at risk may be determined be measuring the MBL level before treatment and only subjecting those to treatment whose MBL level is below a predetermined level.

The fusion protein is administered in suitable dosage regimes, in particularly it is usually administered at suitable intervals, eg. once or twice a week-during chemotherapy.

Normally from 1-100 mg is administered per dosage, such as from 2-10 mg, mostly from 5-10 mg per dosage. Mostly about 0.1 mg/kg body weight is administered.

Furthermore, an aspect of the present invention is the use of a recombinant composition according to the present invention in a kit-of-parts further comprising another medicament. In particular the other medicament may be an anti-microbial medicament, such as antibiotics.

Concerning miscarriage, it has been reported that the frequency of low plasma levels of MBL is increased in patients with otherwise not explained recurrent miscarriages, which is the background for lowering of the susceptibility to miscarriage by a reconstitution of the MBL level by administration of recombinant MBL in these cases.

As to the nature of compounds of the invention, it appears, that in its broad aspect, the present invention relates to compounds which are able to act as opsonins, that is, able to enhance uptake by macrophages either through direct interaction between the compound and the macrophage or through mediating complement deposition on the target surface.

### EXAMPLES

### Example 1

Plasmidcloning of FCNMBL-r1, r2, -r3, -r4, -r5, -r6 and -r7.

1.1 Summary

A series of plasmids were constructed for the expression in mammalian cells of protein fusions between recombinant human mannose-binding lectin 2 gene (rhMBL) and human ficolin 2 (FCN2). The vector is derived from a high-copy-number ColE1-based plasmid and is designed to allow protein expression in mammalian systems. The fusion protein expressions are driven by the human cytomegalovirus (CMV) immediate early promoter to promote constitutive expression. Selection is made possible in bacteria by the ampicillin-resistance gene under control of the prokaryotic β-lactamase promoter. The neomycin-resistance gene is driven by the SV40 early promoter, which provides stable selection with G418 in mammalian cells.

1.2 Constructs and Experimental Work

In order to express fusion proteins between Ficolin2 and MBL we have designed and constructed a series of plasmids. The new recombinant plasmids are based on the previously cloned pcDNA2001-cintMBLcDNA. This plasmid contains a synthetic intron together with the cDNA for human MBL.

The following fusions were designed (underlined font indicates FCN2 part—italics indicate MBL part of the fusion protein.)

FCN2 MBLr1 (SEQ ID NO:118):

FCN2 (signalseq+collagen+“hinge” to ficolin dom aa131) MBL (from aa129 carbohyd.bind dom.)

MELDRAVGVLGAATLLLSFLGMAWALQAADTCPEVKMVGLEGSDKLTIL-
RGCPGLPGAPGDKGEAGTNGKRGERG
PPGPPGKAGPPGPNGAPGEPQPCLTGPRTCKDLLDRGHFLSGWHTIYLP-
DCRPLTFSLGKQVGNKFFLTNGEIMT
FEKVKALCVKFQASVATPRNAAENGAIQNLIKEEAFLGITDEKTEGQFV-
DLTGNRLTYTNWNEGEPNNAGSDEDC
VLLLKNGQWNDVPCSTSHLAVCEFPI

FCN2 MBLr2 (SEQ ID NO:119):

FCN2 (signalseq+collagen+“hinge”+part of flcolin-dom. containing pred. coil-coil to aa207) MBL (from aa129 carbohyd.bind dom.)

MELDRAVGVLGAATLLLSFLGMAWALQAADTCPEVKMVGLEGSDKLTIL-
RGCPGLPGAPGDKGEAGTNGKRGERG
PPGPPGKAGPPGPNGAPGEPQPCLTGPRTCKDLLDRGHFLSGWHTIYLP-
DCRPLTVLCDMDTDGGGWTVFQRRVD
GSVDFYRDWATYKQGFGSRLGEFWLGNDNIHALTAQGTSELRVDLVDFE-
DNYQFAKLTFSLGKQVGNKFFLTNGE
IMTFEKVKALCVKFQASVATPRNAAENGAIQNLIKEEAFLGITDEKTEG-
QFVDLTGNRLTYTNWNEGEPNNAGSD
EDCVLLLKNGQWNDVPCSTSHLAVCEFPI

FCN2 MBLr3 (SEQ ID NO: 120):

FCN2 (signalseq+collagen to aa92) MBL (from aa101 coil-coil+carbohyd.bind dom.)

MELDRAVGVLGAATLLLSFLGMAWALQAADTCPEVKMVGLEGSDKLTIL-
RGCPGLPGAPGDKGEAGTNGKRGERG
PPGPPGKAGPPGPNGAPPDGDSSLAASERKALQTEMARIKKWLTFSLG-
KQVGNKFFLTNGEIMTFEKVKALCVKF
QASVATPRNAAENGAIQNLIKEEAFLGITDEKTEGQFVDLTGNRLTYTN
WNEGEPNNAGSDEDCVLLLKNGQWND
VPCSTSHLAVCEFPI

FCN2 MBLr4 (SEQ ID NO: 121):

FCN2 (signalseq+part of collagen to cons.K at aa93) MBL (from cons.K at aa77 rest of collagen+coil-coil+carbohyd.bind dom.)

MELDRAVGVLGAATLLLFFLGMAWALQAADTCPEVKMVGLEGSDKLTIL-
RGCPGLPGAPGDKGEAGTNGKRGERG
PPGPPGKLGPPGNPGPSGSPGPKGQKGDPGKSPDGDSSLAASERKALQT-
EMARIKKWLTFSLGKQVGNKFFLTNG
EIMTFEKVKALCVKFQASVATPRNAAENGAIQNLIKEEAFLGITDEKTE-
GQFVDLTGNRLTYTNWNEGEPNNAGS
DEDCVLLLKNGQWNDVPCSTSHLAVCEFPI

FCN2 MBLr5 (SEQ ID NO: 122):

FCN2 (signalseq+part of collagen to cons.G at aa69) MBL (from cons.G at aa.64 rest of collagen (containing “kick”)+coil-coil+carbohyd.bind dom.)

MELDRDAVGVLGAATLLLSFLGMAWALQAADTCPEVKMVGLEGSDKLTI-
LRGCPGLPGAPGDKGEAGTNGQGLRGL
QGPPGKLGPPGNPGPSGSPGPKGQKGDPGKSPDGKSSLAASERKALQTE-
MARIKKWLTFSLGKQVGNKFFLTNGE
IMTFEKVKALCVKFQASVATPRNAAENGAIQNLIKEEAFLGITDEKTEG-
QFVDLTGNRLTYTNWNEGEPNNAGSD
EDCVLLLKNGQWNDVPCSTSHLAVCEFPI

FCN2 MBLr6 (SEQ ID NO: 123):

MBL (replaced MBL collagen (aa.41 to aa 99)+coil-coil+carbohyd.bind dom.) FCN2 (inserted collagen aa.54 to aa.92)

MSLFPSLPLLLLSMVAASYSETVTCEDAQKTCPAVIACSSPGCPGLPGA-
PGDKGEAGTNGKRGERGPPGPPGKAG
PPGPNGAPSPDGDSSLAASERKALQTEMARIKKWLTFSLGKQVGNKFFL-
TNGEIMTFEKVKALCVKFQASVATPR
NAAENGAIQNLIKEEAFLGITDEKTEGQFVDLTGNRLTYTNWNEGEPNN-
AGSDEDCVLLLKNGQWNDVPCSTSHL
AVCEFPI

FCN2 MBLr7 (SEQ ID NO: 124):

MBL (signal seq. to aa.25)FCN2 (collagen to aa93) MBL (from aa100 coil-coil+carbohyd.bind dom.)

MSLFPSLPLLLLSMVAASYSALQAADTCPEVKMVGLEGSDKLTILRGCP-
GLPGAPGDKGEAGTNGKRGERGPPGP
PGKAGPPGPNGAPSPDGDSSLAASERKALQTEMARIKKWLTFSLGKQVG-
NKFFLTNGEIMTFEKVKALCVKFQAS
VATPRNAAENGAIQNLIKEEAFLGITDEKTEGQFVDLTGNRLTYTNWNE-
GEPNNAGSDEDCVLLLKNGQWNDVPC
STSHLAVCEFPI

Parental Plasmids Used for All Constructions:

pcDNA2003-cintMBLcDNA

Invitrogen Genestorm clone RG000632 (Cat. No. H-K1000 Invitrogen).

Constructions were done by recombination using the BD In-Fusion™ PCR Cloning Kit form BD (Cat. No. 631774). The BD In-Fusion Kit allows the cloning of PCR products based only on 2×15 bp homology between vector and end of the PCR product. Ligase, or phosphatase are unnecessary when cloning with this kit. The In-Fusion enzyme captures the DNA fragment ends and fuses the insert to the vector.

Primers Used for the PCR Reactions are Shown in Table 1.

PCR Reactions and Linearization of Vector for Recombination

PCR reactions were done on plasmid “Genestorm RG000632” batch N135-15C digested with Bstz17I (N135-20B). Primers pairs were used as described below. Kit for PCR reactions: PfuUltra™ Hotstart PCR Master Mix Stratagene #600630. The PCR reaction tubes were run on the BioRAD i-cycler using the temperature profile shown in table 2.

For the recombination reactions the vector pcDNA2001-cintMBLcDNA was linearized by restriction enzyme digestion with the enzymes listed below.

FCN2 MBLr1:

PCR using primers: Pr1-xho-MBLFCN+Pr4-Xmn-FCNMBL-rev (product 463 bp)

Digest of pcDNA2001-cintMBLcDNA: XhoI+XmnI (partial)

FCN2 MBLr2:

PCR USING PRIMERS: Pr1-xho-MBLFCN+Pr5-Xmn-FCNMBL-rev

Digest of pcDNA2001-cintMBLcDNA: XhoI+XmnI (partial)

FCN2 MBLr3:

PCR USING PRIMERS: Pr1-xho-MBLFCN+Pr6-b-Bsp-FCNMBL-rev

Digest of pcDNA2001-cintMBLcDNA: XhoI+BspEI

FCN2 MBLr4:

PCR USING PRIMERS: Pr1-xho-MBLFCN+Pr2-apa-FCNMBL-rev

Digest of pcDNA2001-cintMBLcDNA: XhoI+ApaI

FCN2 MBLr5:

PCR USING PRIMERS: Pr1-xho-MBLFCN+Pr3-apa-FCNMBL-rev

Digest of pcDNA2001-cintMBLcDNA: XhoI+ApaI

FCN2 MBLr6:

PCR USING PRIMERS: Pr8-BstAP-MBLFCN+Pr6-b-Bsp-FCNMBL-rev

Digest of pcDNA2001-cintMBLcDNA: partial BstAPI+BspEI

FCN2 MBLr7:

PCR USING PRIMERS: Pr7-Alw-MBLFCN+Pr6-b-Bsp-FCNMBL-rev

Digest of pcDNA2001-cintMBLcDNA:partial AlwNI+BspEl

In-Fusion PCR Recombination Reactions

In-Fusion PCR recombination reactions were set up using approx. 50-100 ng of Quiagen Minelute purified PCR products together with 50-100 ng of Quiagen Minelute purified linearized pcDNA2001-cintMBLcDNA.

1/10 of the recombinaton reactions were transformed into MAX efficiency DH5α Competent Cells (Invitrogen Cat. No. 18258-012). 1/10 and 9/10 from each transformation were spread on separate LB plates containing 200 ug/ml ampicillin.

Plates were incubated at 37° C. overnight.

Screening for positive clones: At least 6 colonies from each experimental plate were picked for miniprep plasmid DNA isolation. To determine the presence of insert, DNA was analyzed by restriction digest analysis with the enzyme PstI. Three individual positive clones from each reaction were chosen for further work.

Restriction Analysis

In order to verify the selected individual recombinant plasmids after the primary screen we performed an intensive restriction enzyme digestion analysis on the plasmid DNA isolated.

Plasmid DNA of the recombinants were digested with the enzyme shown in table 3.

The expected fragments are also listed in the table. All recombinant clones tested exhibited the expected pattern. Digestion with EcoRI was not as predicted. An additional fragment was observed both in digestion of the recombinant as well of the parental plasmid. The discrepancy can be explained by an additional EcoRI site on the parental plasmid.

Results

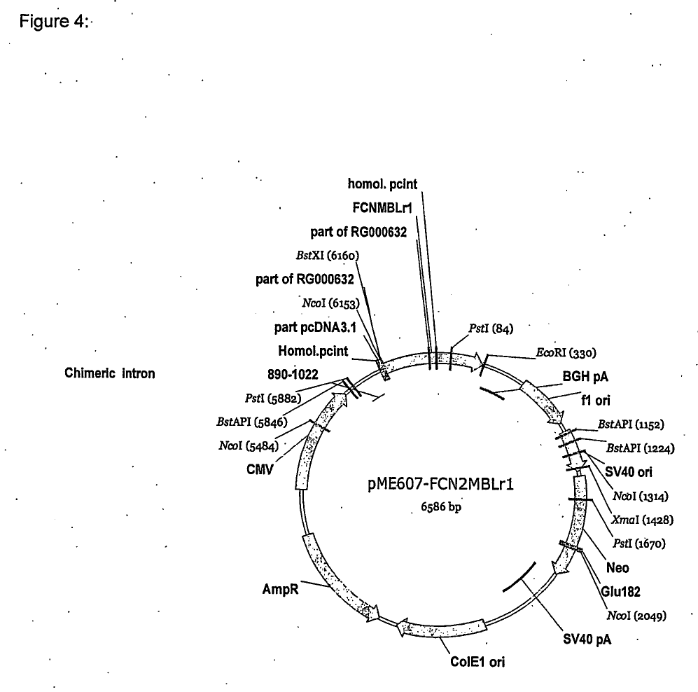

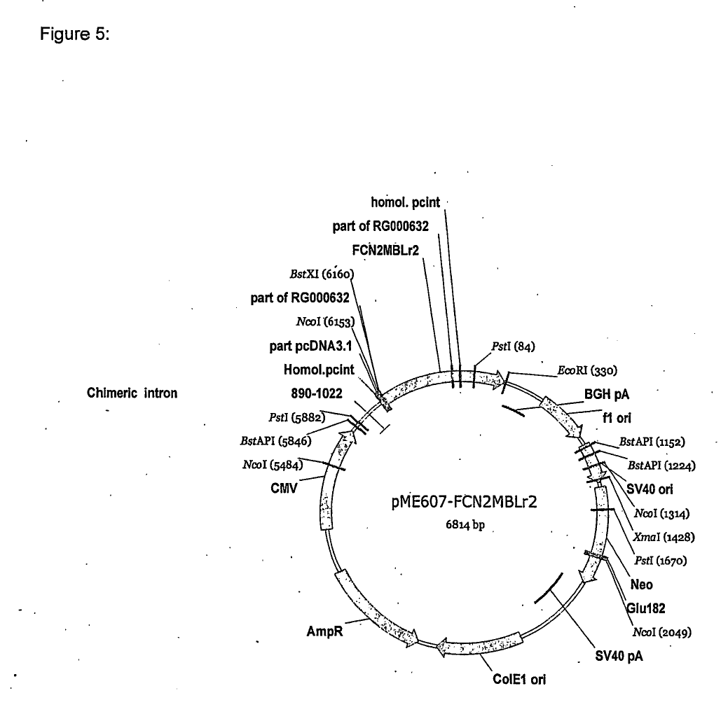

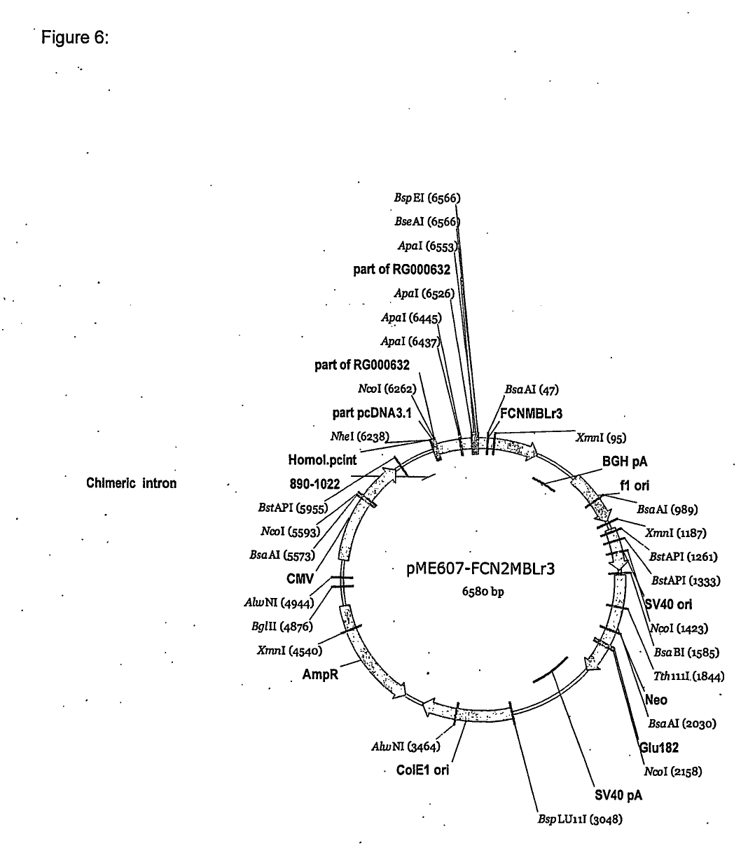

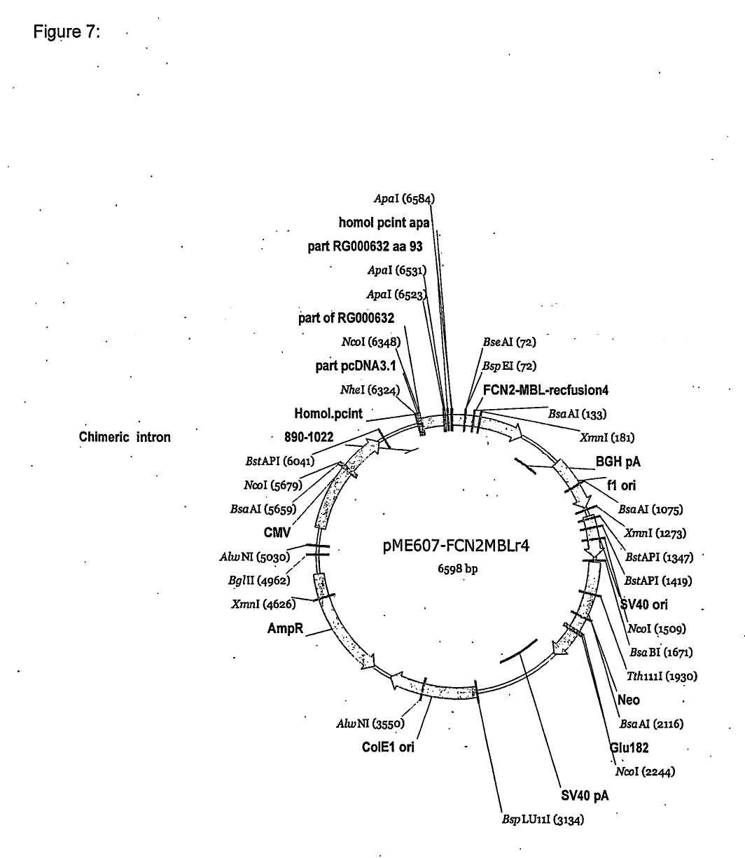

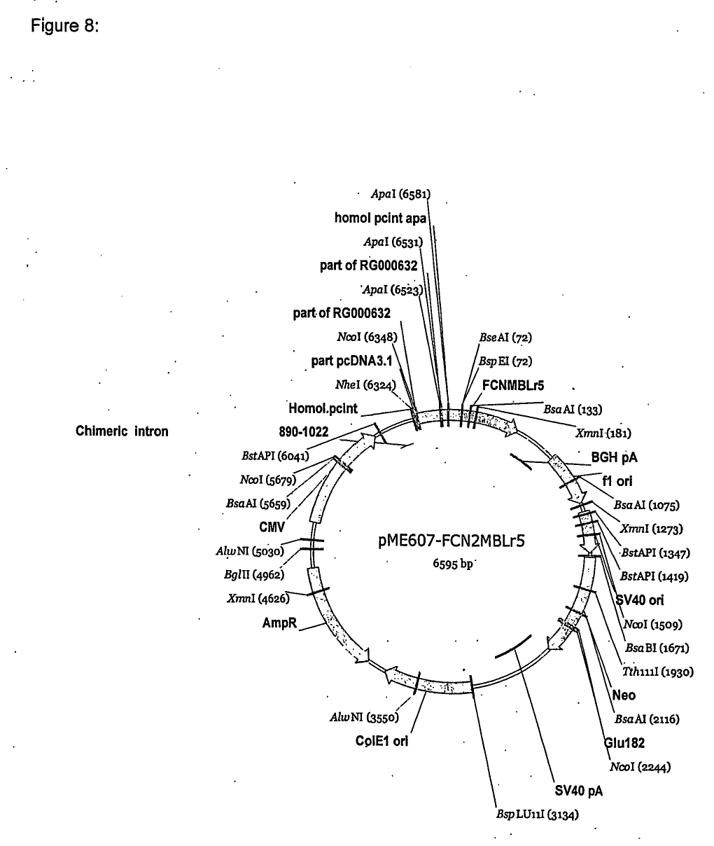

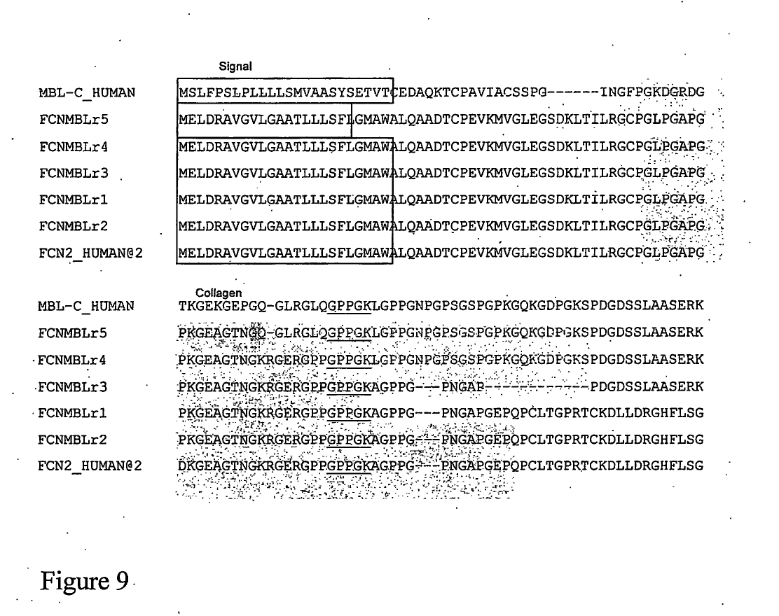

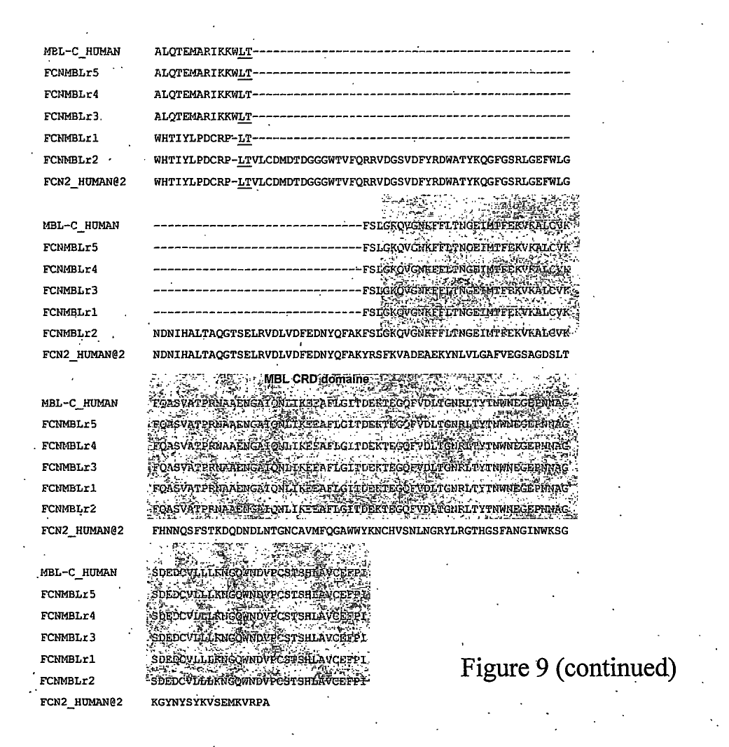

Recombinant plasmids obtained are shown schematically in FIGS. 4-8 for constructs r1, -r2, -r3, -r4, and -r5.

### Example 2

Experiments with Transient Expression of Recombinant Fusion Proteins of Human MBL and Human FCN2

2.1 Summary

We report the expression of recombinant human fusion proteins FCNMBLr1, FCNMBLr4, FCNMBLr5 and MBL in HEK293 and Per.C6 cells. We found that the cell lines in the transient transfection experiment were able to produce at least the fusion proteins FCNMBLr4 and FCNMBLr5 assembled in active oligomeres with a structure primarly similar to MBL-oligomer forms 3 and 4. The fusion proteins FCNMBLr4 and FCNMBLr5 behaved like MBL upon binding to a carbohydrate surface and upon activating the complement cascade.

2.2 Introduction

The aim of the studies was to elucidate the possibility of creating a hybrid protein consisting of the collagen part of human ficolin 2 and the human mannose binding lectin (MBL). Furthermore we wished to clarify if such molecules would still posses the ability to bind to complex carbohydrate structures and still are able to activate complement.

Two eukaryotic cell lines of human origin HEK293 and Per.C6 were used as host cell lines for transient transfections with the respective expression plasmids. Transcription was driven by the CMV-IE promoter enhancer.

2.3 Experimental

Material and Methods

Plasmids Used for the Transfection Experiments

pME607-FCNMBL-r1, -r2, -r3, -r4, -r5, -r6 and -r7 (described in example 1)

Origin of Cells Used

PerC6 cells were obtained from Crucell.

HEK 293 Freestyle cells were obtained from InVitrogen.

Culture Media

PerC6 cells were cultured at 37° C. in 10% (vol/vol) CO₂ maintained as monolayers in serum free medium.

HEK 293 Freestyle were cultured at 37° C. in 8% (vol/vol) CO₂ maintained as suspension in an Invitrogen Freestyle medium.

Transfections and Harvest of Media

Per.C6 cells in serum containing medium were transfected with the DNA-using the transfection reagent Lipofectamine. One day after transfection the medium was replaced with serum free medium.

HEK293 cells in serum free Freestyle medium were transfected with the DNA using the transfection reagent 293fect. The medium was collected after approximately 4 days of incubation after transfection.

Quantification of MBL

Recombinant MBL assay (TRIFMA) using Mannan coated plates or mAb-131-01 coated plates. For quantification of MBL, time-resolved immunofluorometry was carried out.

SDS-PAGE and Western Blot Analysis

SDS-PAGE with subsequent electrophoretic transfer of proteins to polyvinylidene diflouride membranes and detection of MBL using monoclonal anti-MBL antibody was carried out.

C4 Assay

The assay is designed to measure MBL and rMBL abilities to initiate the MBL Lectin-pathway of the complement system. MBL associated serine protease (MASP 2) associated with MBL cleaves the complement factor C4 releasing C4a and C4b. The C4b deposition on the Mannan coated ELISA plates is detected with biotin labelled antibodies against C4b and Europium labelled Streptavidin.

2.4 Results

In the experiments described herein we were able to express FCN2 MBLr4, FCN2 MBLr5 and MBL transiently in both HEK293 and Per.C6 cells under serum free conditions.

Oligomeric Form of the Fusion Proteins

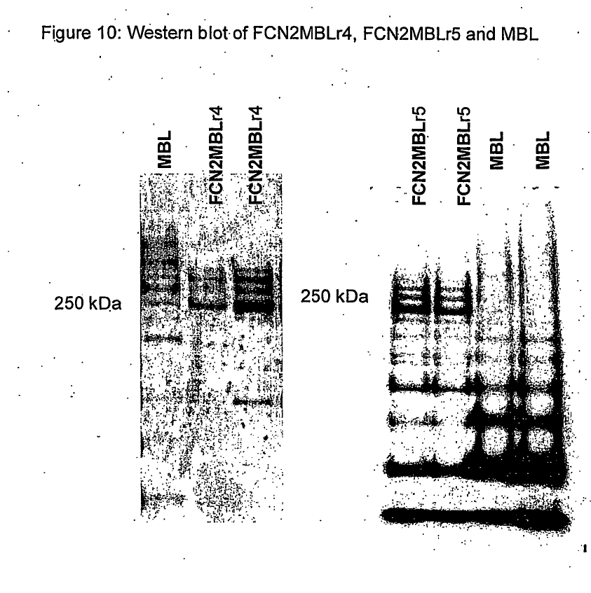

The oligomeric forms of the fusion protein were examined by non-reducing denaturizing SDS PAGE followed by a western blot. The detecting antibody recognizes the CDR part of MBL (and maybe part of the coil-coil region). The results are shown in FIG. 10. It is evident from the figure that the most prominent form of the fusion proteins FCN2 MBLr4 and FCN2 MBLr5 is approximately 250 kDa corresponding to a 3- or 4-mere of subunits consisting of 3 single protein chains (24 kDa). The appearance of the oligomeric form was independent of the host cells used. MBL was produced in a wide range of oligomeric forms.

Binding Properties

The fusion proteins were tested for functionality of the MBL carbohydrate binding domain by binding to a mannan surface and detection with an antibody that recognizes the CDR part of MBL (and maybe part of the coil-coil region). The results are shown in table 4. It can be concluded that FCN2 MBLr4 and FCN2 MBLr5 were expressed just as well as MBL in the host cells and that the fusion proteins bind to a mannan surface.

MASP-2 Binding and C4 Cleavage

The fusion proteins were further tested for the capacity to bind MASP-2 and for activating the serine protease of MASP-2. This was done by measuring cleavage of the MASP-2 substrate complement factor C4 upon binding of the fusion protein to a mannan surface. Results are shown in table 5. It can be concluded from these results that the fusion proteins FCN2 MBLr4 and FCN2 MBLr5 preserved the ability to bind and activate MASP-2.

Discussion

The results described herein clearly demonstrate that it is possible to construct fusion proteins of FCN 2 and MBL with the following properties:

1. The oligomeric structure of the fusion proteins is more simple than that of the MBL protein.

2. The fusion proteins keep the essential property of MBL activation of the complement cascade upon binding to a dense carbohydrate structure.

TABLE 1

Primers used for the PCR reactions

| | | Primer part of | |
|---|---|---|---|
| Primer | | pcDNA2001- | Primer part of |
| name | DNA Sequence of Primer | cintMBLcdna | RG000632 |
| Pr1-xho- | ataggctagcctcgaagctcgcc- | ataggctagcctcga | agctcgcccttcacc |
| MBLFCN | cttcaccatggagctggacag | | atggagctggacag |
| Pr2-apa- | Ccaactttccaggggggcccgggg | g(replaced) | ggaaagagagga- |
| FCNMBL- | ggccacgttctcctctctttcc | ggcccccctggaaag | gaacgtggcccccc |
| rev | | ttgg | |
| Pr3-apa- | Ccaactttccaggggggccctgta | Caagggctca- | cccaaggga- |
| FCNMBL- | agcctctgagcccttgtccattgg | gaggctta- | gaggcaggcac- |
| rev | tgcctgcctctcccttggg | cagggcccccctgga | caatgga |
| | | aagttgg | |
| Pr4-Xmn- | Tggtcaggaagaacttgttccca- | Ttctctctgggcaaa- | cctgcccgactgcc |
| FCNMBL- | acttgtttgcccagagagaaagt- | caagttgggaa- | ggcccctgact |
| rev | caggggccggcagtcgggcagg | caacttcttcctgacc | |
| | | a | |
| Pr5-Xmn- | Tggtcaggaagaacttgttccca- | Ttctctctgggcaaa- | ggactttgagga- |
| FCNMBL- | acttgtttgcccagagagaactt- | caagttgggaa- | caactac- |
| rev | agcaaactggtagttgtcctcaa | caagttcttcctgacc | cagtttgctaag |
| | agtcc | a | |
| Pr6-b-Bsp- | Gactatcaccatccggaggtgctc- | ccggatggtgatagt | ggac- |
| FCNMBL- | cgttgggcccaggtggtcc | | cacctgggcccaac |
| rev | | | ggagcacct |
| Pr7-Alw- | Cagcgtcttactcagctctccagg- | cagcgtcttactcag | ctctccaggcggca |
| MBLFCN | cggcagacacctgtcc | | gacacctgtcc |
| Pr8-BstAP- | Agacctgccctgcagtgattgcct- | Agacctgccctgca | ggctgtccggggct |
| MBLFCN | gtagctctccaggctgtccggggc | gtgattgcctgtagct | gcctggggcccc |
| | tgcctggggcccc | ctcca | |

Sequence typed In bold shows the 15 bp homology needed for the recombination into the vector.

TABLE 2

| Cycle | times | step | Temp | Time |
|---|---|---|---|---|
| 1 | 1x | 1 | 95° | 21 min |
| 2 | 30x | 1 | 95° | 0 min 30 sec |
| | | 2 | 72° (67.5° for r2) | 0 min 30 sec |
| | | 3 | 72° | 1 min |
| 3 | 1x | 1 | 72° | 10 min |
| 4 | 1x | 1 | 4° | ∞ |

TABLE 3

| pcDNA2001- | pME607- | pME607- | pME607- | pME607- | pME607- |
|---|---|---|---|---|---|
| cintMBLcDNA | FCN2MBLr1 | FCN2MBLr2 | FCN2MBLr3 | FCN2MBLr4 | FCN2MBLr5 |
| PstI | PstI | PstI | PstI | PstI | PstI |
| 4212 | 4212 | 4212 | 4212 | 4212 | 4212 |
| 1586 | 1586 | 1586 | 1486 | 1586 | 1586 |
| 405 | 788 | 1016 | 782 | 800 | 797 |
| 375 | | | | | |
| EcoRI | EcoRI | EcoRI | EcoRI | EcoRI | EcoRI |
| 5810 | 6586 | 6814 | 6580 | 6598 | 6595 |
| 768 | | | | | |
| XmaI | XmaI | XmaI | XmaI | XmaI | XmaI |
| 6578: | 6586 | 6814 | 6580 | 6589 | 6595 |
| BstXI | BstXI | BstXI | BstXI | BstXI | BstXI |
| undigested | 6586 | 6814 | 6580 | 6598 | 6595 |
| BstAPI | BstAPI | BstAPI | BstAPI | BstAPI | BstAPI |
| 4622 | 4622 | 4622 | 4622 | 4622 | 4622 |
| 1469 | 1892 | 2120 | 1886 | 1904 | 1901 |
| 415 | 72 | 72 | 72 | 72 | 72 |
| 72 | | | | | |
| NcoI | NcoI | NcoI | NcoI | NcoI | NcoI |
| 3435 | 3435 | 3435 | 3435 | 3435 | 3435 |
| 2408 | 1747 | 1975 | 1741 | 1759 | 1756 |
| 735 | 735 | 735 | 735 | 735 | 735 |
| | 669 | 669 | 669 | 669 | 669 |

TABLE 4
MBL binding to mannan measured by TRIFMA

| | | μg MBL equivalents/ |
|---|---|---|
| | | ml |
| FCN2MBLr5 | HEK293 | 0.689 |
| FCN2MBLr5 | HEK293 | 0.764 |
| FCN2MBLr1 | HEK293 | 0.874 |
| MBL | HEK293 | 0.457 |
| FCN2MBLr4 | HEK293 | 0.851 |
| MBL | HEK293 | 1.885 |
| FCN2MBLr5 | Per.C6 | 0.296 |
| MBL | Per.C6 | 0.271 |
| FCN2MBLr4 | Per.C6 | 0.077 |
| FCN2MBLr4 | Per.C6 | 0.091 |
| FCN2MBLr4 | Per.C6 | 0.089 |
| FCN2MBLr4 | Per.C6 | 0.035 |
| MBL | Per.C6 | 0.092 |

TABLE 5
C4 activity of the fusion proteins

| | Cells | Aktivitet +/− |
|---|---|---|
| pME607-FCNMBLr5 clone 1 | HEK293 | + |
| pME607-FCNMBLr5 clone 5 | HEK293 | + |
| pME607-FCNMBLr4 clone 2 | HEK293 | +/+ (after purification) |
| pcDNA2001-cintMBLcDNA | HEK293 | + |
| pME607-FCNMBLr5 clone 5 | Per.C6 | + |
| pME607-FCNMBLr4 clone 2 | Per.C6 | −/(+) (after purification) |
| (maxi | | |

1. A fusion protein comprising
i) a first polypeptide sequence derived from a lectin-complement pathway activating protein or a functional homologue at least 70% identical to said lectin-complement pathway activating protein, wherein said first polypeptide sequence is capable of activating protein, wherein said first polypeptide sequence is capable of activating the lectin-complement pathway; and
ii) a second polypeptide sequence derived from a collectin or a functional homologue at least 70% identical to said collectin, wherein said second polypeptide sequence is capable of associating with one or more carbohydrates;
wherein said complement activating protein is not a collectin.

2. (canceled)

3. The fusion protein according to claim 1, wherein said first polypeptide sequence is capable of associating with at least one MASP protein.

4. The fusion protein according to claim 1, wherein said first polypeptide sequence is capable of associating with a MASP protein selected from the group consisting of MASP-1, MASP-2 and MASP-3 or functional homologues or variants hereof.

5. The fusion protein according to claim 1, wherein the complement activating protein is a ficolin.

6. The fusion protein according to claim 5, wherein the ficolin is selected from the group consisting of L-ficolin, H-ficolin and M-ficolin.

7. The fusion protein according to claim 5, wherein the ficolin is L-ficolin.

8. (canceled)

9. The fusion protein according to claim 1, wherein the first polypeptide sequence comprises the collagen-like domain of a ficolin or a functional homologue or variant thereof.

10. (canceled)

11. The fusion protein according to claim 1, wherein the first polypeptide sequence comprises the cysteine-rich region of a ficolin or a functional homologue thereof.

12. (canceled)

13. The fusion protein according to claim 1, wherein the first polypeptide sequence comprises the cysteine-rich region and the collagen-like domain of a ficolin or a functional homologue or variant thereof.

14. (canceled)

15. The fusion protein according to claim 1, wherein the first polypeptide sequence comprises amino acids 1-77 of the L-ficolin sequence of FIG. 1 (SEQ ID. NO 125).

16. (canceled)

17. The fusion protein according to claim 1, wherein the collectin is selected from the group consisting of MBL (mannose-binding lectin), SP-A (lung surfactant protein A), SP-D (lung surfactant protein D), BK (or BC, bovine conglutinin) and CL-43 (collectin-43).

18. The fusion protein according to claim 17, wherein the collectin is MBL.

19. (canceled)

20. The fusion protein according to claim 1, wherein the second polypeptide sequence comprises the CRD domain of a collectin or a functional homologue or vaiant thereof.

21. The fusion protein according to claim 1, wherein the second polypeptide sequence comprises the CRD domain of MBL.

22. The fusion protein according to claim 1, wherein the second polypeptide sequence comprises the neck region of MBL.

23. The fusion protein according to claim 1, wherein the second polypeptide sequence comprises the collagen-like domain of MBL.

24. The fusion protein according to claim 1, wherein the second polypeptide sequence comprises the neck region and the CRD domain of MBL.

25. The fusion protein according to claim 1, wherein the second polypeptide sequence comprises the collagen-like domain, the neck region and the CRD domain of MBL.

26. The fusion protein according to claim 1, wherein the second polypeptide sequence comprises amino acids 80-228 of the MBL sequence shown in FIG. 2 (SEQ ID. No 126).

27. The fusion protein according to claim 1, wherein the fusion protein comprises the the cysteine-rich region and the collagen-like domain of L-ficolin and the CRD domain of MBL.

28. The fusion protein according to claim 1, wherein the fusion protein comprises the cysteine-rich region of L-ficolin and the collagen-like domain, the neck region and the CRD domain of MBL.

29. The fusion protein according to claim 1, wherein the fusion protein comprises the amino acid sequence as defined by the sequence shown in FIG. 3 (SEQ ID. NO. 127), or a functional homologue at least 70% identical thereto.

30. The fusion protein according to claim 1, wherein the fusion protein consists of the amino acid sequence as defined by the sequence shown in FIG. 3 (SEQ ID. NO 127).

31. An isolated nucleic acid comprising a nucleotide sequence encoding the fusion protein according to claim 1.

32. A vector comprising the nucleic acid sequence according to claim 31.

33. A cell comprising the vector according to claim 32.

34.-36. (canceled)

37. A method of prevention and/or treatment of an infection in an individual in need thereof comprising administering to said individual an effective amount of the fusion protein according to claim 1.

38. (canceled)

39. The method according to claim 37, wherein the individual is a human being.

40. The method according to claim 37, wherein the individual is a human being suffering from an increased risk of acquiring an infection.

41. The method according to claim 37, wherein the individual is a human being with subnormal serum MBL level.

42. The method according to claim 37, wherein the individual is a human being with normal serum MBL level.

43.-48. (canceled)

49. A pharmaceutically acceptable composition for the treatment or prevention of a clinical condition in an individual in need thereof, comprising the fusion protein according to claim 1, and a pharmaceutically acceptable carrier.

50.-51. (canceled)
